(19) United States
(12) Patent Application Publication
(10) Pub. No.: US 2016/0228040 A1
(43) Pub. Date: Aug. 11, 2016

(54) Notification System

(71) Applicant: KONICA MINOLTA, INC., Chiyoda-ku (JP)

(72) Inventors: Yuichi ATARASHI, Hachioji-shi, Tokyo (JP); Takashi OKADA, Hino-shi, Tokyo (JP)

(21) Appl. No.: 15/021,537

(22) PCT Filed: Sep. 5, 2014
(86) PCT No.: PCT/JP2014/073550

§ 371 Date: Mar. 11, 2016

(30) Foreign Application Priority Data
Sep. 13, 2013 (JP) 2013-191162
Sep. 13, 2013 (JP) 2013-191172

(51) Int. Cl. A61B 5/11; A61B 5/00

(52) U.S. Cl. CPC A61B 5/1117; A61B 5/746; A61B 5/7282; A61B 5/0002

(57) ABSTRACT

A notification system that appropriately notifies a healthcare provider of a change in the state of a patient in a sickroom is provided. The notification system includes: a detection unit that generates movement information of a subject according to a change over time; a storage unit that is connected to the detection unit and stores the movement information; and a control unit that receives the movement information from the detection unit, analyzes the movement information, makes a determination on the occurrence of a predetermined event from the result of the analysis, specifies the movement information in a predetermined period including the time when the determination was made, applies a storage priority value to the movement information according to a type of the predetermined event occurred, and transmits the movement information to the storage unit.

### TECHNICAL FIELD

The present invention relates to a notification system that outputs a notification in response to a predetermined call signal and is ideally used for substitution of a nurse call system which is provided in hospitals, healthcare facilities, and the like, for example.

### BACKGROUND ART

A nurse call system is known as a notification system which is broadly used in hospitals. In this nurse call system, when a call button is operated by a patient in a sickroom, for example, a notification module provided in a nurse's station or the like outputs a notification. A healthcare provider such as a nurses or a caregiver present in a nurse's station responds to the notification.

In a conventional general nurse call system, an extension module is provided in each bed of a patient, provided in a sickroom. The extension module has a call button that allows patients to call a healthcare provider. This call button is placed at the hands of a patient, for example. The extension module is communicably connected to a base module. The base module has a notification module and the notification module operates when the call button is pressed.

The base module is provided as a panel on the wall of a nurse's station which is a headquarter, for example. An arbitrary notification module such as a display panel or a speaker, for example, is provided in the panel. When the base module has a function of displaying information and outputting sound as a notification function, information that can identify a patient is displayed on the display panel when the patient operates the call button. Further, the speaker emits a notification sound indicating that the call button has been operated. Moreover, when a PBX module or the like is incorporated into the nurse call system, the base module may send an external notification (call) to a mobile terminal such as PHS using an operation of the call button as a trigger.

In recent years, a configuration in which a camera is combined with a nurse call system has been proposed. This camera is provided in a sickroom, for example. In this configuration, the camera is associated with an extension module. When a call button associated with the extension module is operated, the surroundings of the patient's bed in which the extension module is provided is imaged by the corresponding camera and video information is generated. This video information is transmitted to the base module, and a video based on the video information is displayed on a display monitor provided in the base module. With this configuration, a healthcare provider can visually understand the patient's state when the call button was operated (for example, see Patent Literature 1).

In this way, in the nurse call system, a notification sound is output from the notification module provided in the base module in response to an operation of the call button, and information such as a video is transmitted to the display monitor or the speaker provided in the base module. When the base module is provided in a nurse's station, a healthcare provider or the like can understand a change or the like in the patient's state while sitting in the nurse's station and can take quick countermeasures when a serious change (a serious event) occurs in the patient's body, for example.

### CITATION LIST

### Patent Literature

Patent Literature 1: JP 2008-079800 A

### SUMMARY OF INVENTION

### Technical Problem

As described above, in the conventional nurse call system, a video is displayed when a patient recognizes a change in his or her state and presses the call button. That is, the image displayed on the display monitor shows the state after a serious change occurs in the patient's body. Thus, a healthcare provider viewing the image may be unable to understand which event has caused this situation. For example, when the call button was pressed after a patient fell off a bed, the situation during falling is not displayed on the display monitor. Thus, it is difficult for the healthcare provider to understand how the fall occurred. That is, it may be difficult for the healthcare provider to specify an injured part, and in this case, the healthcare provider cannot take quick countermeasures such as medical aids.

As a method of solving this problem, a method of storing all items of video information and the like imaged and generated by a camera and allowing a healthcare provider to retrieve and specify a video corresponding to a situation in which a serious change occurs in a patient's body when such a change occurs may be used. However, the amount of the video information may be too large in a large hospital having a large number of inpatients. Thus, the volume of a storage device in which the video information is stored may be insufficient and it may be impossible to store the information. Moreover, management of the large amount of video information is very troublesome.

Since such a multi-functional nurse call system as described above eliminates and substitutes an existing nurse call system, it may take a very large cost to introduce such a system.

Therefore, an object of the present invention is to provide a notification system that appropriately notifies a healthcare provider of a change in the state of a patient in a sickroom. For example, the present invention provides a notification system which enables a healthcare provider to understand a patient's state in a period before and after the occurrence time of a change when the change occurs in a patient and a notification is issued.

### Solution to Problem

In order to solve the above problem, a notification system of the present invention includes: a detection unit that generates movement information of a subject according to a change over time; a storage unit that is connected to the detection unit and stores the movement information; and a control unit that receives the movement information from the detection unit, analyzes the movement information, makes a determination on the occurrence of a predetermined event from the result of the analysis, specifies the movement information in a predetermined period including the time when the determination was made, applies a storage priority value to the movement information according to a type of the predetermined event occurred, and transmits the movement information to the storage unit.

For example, one of a plurality of detection unit is connected to the control unit. When a plurality of detection unit is connected to the control unit, the control unit is configured to collectively process the detection information received from the plurality of detection unit.

The “subject” means a person who stays for a predetermined period in a confined space and who is monitored in this space. Examples of the subject include a patient, a caretaker, a person who performs dangerous works, and the like. Here, the “movement information of the subject” is a movement of the subject specified by performing a predetermined process on the detection information generated by the detection unit, for example.

The detection unit may include an arbitrary configuration as long as it has a configuration capable of continuously detecting the motion of a person. Examples of the detection unit include a non-contact detection device capable of detecting the motion without making contact with the subject (patient). Examples of the detection device include an imaging device such as a video camera, a sound recording device such as a digital recorder or an analog recorder, and a moving object detection device such as an ultrasound sensor, a microwave Doppler sensor, or an infrared sensor. At least two of the above-mentioned detection devices may be combined to form the detection unit.

The control unit determines whether a predetermined event has occurred based on the event determination information and the movement information of the subject, specified by the analysis, for example. The event determination information includes information corresponding to the predetermined event. The event determination information is stored in advance in a storage module or a storage unit (hereinafter sometimes referred to as a storage unit or the like) included in the control unit, for example.

The “predetermined event” means an event that is to be noticed when the subject is monitored. The “event” means a change in the subject's state occurring accidentally. Among the state changes, tumbling and falling are set as the “predetermined event,” for example. The “predetermined event” can be appropriately set according to the type of the subject. When the subject is an inpatient, getting up, leaving bed, wandering, and the like in addition to these state changes are set as the “predetermined event.” These state changes correspond to a specific movement of the subject, for example. That is, the information corresponding to the predetermined event can be used as information on the specific movement of the subject.

The “storage priority value” indicates the degree of priority of storage of the movement information of the subject, stored in the storage unit. In other words, the storage priority value indicates the degree of priority of removal, and the movement information is removed preferentially from the information of which “the storage priority value” is lower. The degree of priority of storage is set by correlating a number, for example, with each of the respective predetermined events. The correlation is performed based on a correlation table stored in advance in the storage unit or the like, for example.

In the notification system of the present invention, the control unit performs the specification in the predetermined period including the time when the determination was made. The predetermined period starts a first predetermined period earlier than the determination time and ends a second predetermined period later than the determination time.

A notification system of the present invention further includes notification module configured to output a notification to an external device. The control unit transmits, to the notification module, the movement information of the subject in the predetermined period including the time when the determination was made when it is determined that the predetermined event has occurred.

In the notification system of the present invention, the detection unit is an imaging unit configured to be capable of perform imaging. The imaging unit generates video including the movement information of the subject. The control unit includes a first display module configured to be able to display a video. Moreover, the control unit analyzes the video information to determine the presence of a predetermined movement of the subject as the occurrence of the predetermined event based on a change in a luminance value of a pixel that forms the video. Further, the control unit displays a video imaged by the imaging unit in the predetermined period on the first display module according to the result of the determination.

The imaging unit may include an arbitrary configuration as long as it can generate videos such as moving images or still images. A conventional existing imaging device can be appropriately selected and used as the imaging unit. Examples of the imaging device include a digital camera (a digital still camera, a digital video camera, or the like) which is a general visible-light camera. Moreover, in order to perform imaging during lights-out periods in the night, an imaging device such as an infrared night-vision device (an infrared camera) or a thermography night-vision device (a thermography camera) may be used as the imaging unit. Moreover, the imaging unit may be a visible-light camera having the night-vision function.

A video generated by the imaging unit is displayed on the first display module. This video is displayed when the video information stored in the storage unit is reproduced, for example. In this case, a reproduction unit for reproducing the video information is connected to the control unit. Moreover, the control unit may have a function of reproducing the video information. The first display module is a display monitor provided in a headquarter such as a nurse's station, for example.

In the notification system of the present invention, a video indicating a predetermined movement of the subject is a video imaged by the imaging unit in a predetermined period including the time of the determination, stored in the storage unit.

In the notification system of the present invention, the detection unit is a microwave Doppler sensor that includes a transmitting module that emits microwaves to an area including the chest of the subject and a receiving module that receives reflection waves from the subject as the movement information. The control unit analyzes a respiratory state of the subject based on a Doppler shift of the reflection waves. Moreover, the control unit determines whether the respiratory state has reached a predetermined respiratory state as the occurrence of the predetermined event based on the result of the analysis. Further, the control unit transmits control information of the notification module when the predetermined respiratory state is reached.

In the notification system of the present invention, the control unit removes the movement information of the subject stored in the storage unit under a predetermined condition. The removal is executed in ascending order of the storage priority values.

In the notification system of the present invention, the control unit performs the removal when a predetermined period has elapsed from the storage in the storage unit as the predetermined condition. Alternatively, the control unit performs the removal when an available volume of the storage unit is lower than a predetermined value as the predetermined condition.

The notification system according to the present invention includes a transceiving unit configured to be able to transmit information to at least one mobile communication terminal. The control unit transmits information based on the occurrence of the predetermined event specified by the determination to the mobile communication terminal via the transceiving unit.

In the notification system of the present invention, the mobile communication terminal includes a second display module configured to be able to display information based on the movement information. The control unit transmits the storage priority value and the information based on the movement information corresponding to the predetermined event specified by the determination to the mobile communication terminal via the transceiving unit. The mobile communication terminal displays the information on the second display module.

The mobile communication terminal is a device capable of performing wireless communication. Examples of a communication form of the wireless communication include an ad-hoc mode and an infrastructure mode. A combination of these communication forms may be used as the communication form of the wireless communication. The “ad-hoc mode” means a communication form in which a plurality of terminals can communicate with each other in parallel. Moreover, the “infrastructure mode” means a communication form in which communication is performed using one terminal or device as a base module and the other terminal as an extension module. Examples of the mobile communication terminal include a device having a display monitor corresponding to the second display module. A specific example of this device includes a PDA, a tablet terminal, a mobile phone, a smartphone, and the like.

In this case, the control unit can specify a subject corresponding to the video and transmit the movement information of the subject to a specific mobile communication terminal. This specification is performed based on the movement information generated by the detection unit and predetermined correlation information between the detection unit and the subject.

The notification system according to the present invention includes a transceiving unit configured to be able to transmit a video to at least one mobile communication terminal. The mobile communication terminal includes a second display module configured to be able to display the video. The control unit transmits alive video of the subject being imaged by the imaging unit to the mobile communication terminal via the transceiving unit when a predetermined movement of the subject has occurred. The mobile communication terminal is configured so that a terminal operator who operates the mobile communication terminal can make communication or a telephone call with the subject while viewing the live video by performing a predetermined operation.

In the notification system of the present invention, the subject and the mobile communication terminal are correlated and the transceiving unit is configured to be able to transmit information to a specific mobile communication terminal. The detection unit is correlated with the subject. Upon receiving the movement information of the subject from the detection unit, the control unit specifies the subject based on the correlation between the detection unit and the subject. Further, the control unit specifies the mobile communication terminal to which information is to be transmitted based on the correlation between the subject and the mobile communication terminal. After that, the control unit transmits the movement information of the subject to the specified mobile communication terminal via the transceiving unit.

A notification system of the present invention is provided with a notification unit. The notification unit includes an operating module configured to be able to input operations. The notification unit also includes a notification module disposed at a position separated from the operating module and configured to be able to output a notification to an external device. The notification unit is configured such that the notification module operates to output a notification to the external device in response to a first signal for operating the notification module, transmitted from the operating module when an operator inputs an operation. The notification system is further provided with a detection unit that generates movement information of the operator according to a change over time. The notification system is further provided a control unit that receives the movement information from the detection unit, analyzes the movement information, and makes a determination on the occurrence of a predetermined event from the result of the analysis. The notification system is further provided with a relay unit that is provided between the operating module and the notification module and is configured to allow the notification module and the control unit to communicate with each other, the relay unit transmitting the first signal to the notification module. The control unit transmits a second signal to the relay unit when the determination is executed. The relay unit transmits a third signal for operating the notification module to the notification module in response to the second signal.

The “notification unit” may be an arbitrary element as long as it has a configuration that outputs a notification to an external device in response to an input operation. The notification system of the present invention can be formed by appropriately selecting and using a conventional existing notification unit and combining a relay unit with the notification unit. Moreover, the connection form between the operating module and the notification module included in the notification unit may be wireless connection or cable connection.

The “operator” means a person who uses the operating module among the subjects. Those mentioned in relation to the subject can be appropriately applied to the operator. Examples of the operator include a patient, a caretaker, a person who performs dangerous works, and the like. If the place where the detection unit is provided is a sickroom, the operator is a patient, for example.

The relay unit has a configuration capable of transmitting a “third signal” for operating the notification module by being controlled by the control unit. The relay unit may have a configuration capable of transmitting signals output from one or both of the notification module and the operating module to the control unit. When this function is executed, the “first signal” is regarded as the “third signal.”

The relay unit has the function of one or both of a path switcher and a signal converter, for example. Here, when the “second signal” includes the “third signal,” the relay unit functions as the path switcher. When this function is executed, a path of the relay unit connected to the notification module is connected. This connection may be performed by being controlled by the control unit and may be performed according to a connection control signal or the like included in the “second signal.” As a result, the “third signal” transmitted from the control unit reaches the notification module via the relay unit.

Moreover, when the “second signal” includes a “signal for transmitting the “third signal” to the relay unit,” the relay unit functions as the signal converter. When this function is executed, the relay unit receives the “second signal” from the control unit and performs processes such as analysis, determination, conversion, and the like on the signal to generate the third signal. Further, the relay unit transmits the generated “third signal” to the notification module. When the information for identifying the detection unit is associated with the “second signal,” the relay unit functions as the signal converter and this information is analyzed. With this analysis, it is possible to identify the detection unit from which the “second signal” was transmitted. The relay unit transmits a specific control signal to the notification module together with the “second signal,” for example, based on the identification result. As a result, specific display control is performed on the first display module provided in the notification module based on the control signal.

The “specific display control” means control of specifying the information of the operator specified by the control signal and displaying the information. According to an example of this control, the LED corresponding to the operator specified by the control signal may be turned on and off. In this case, the first display module is configured to include a plurality of LEDs provided so as to correspond to the names of operators. When the first display module is a display monitor, the first display module displays information on the name or the like of the operator as a character video.

The notification system of the present invention includes: a first communication unit provided in the relay unit; and a second communication unit provided in the detection unit or the control unit. The first communication unit and the second communication unit communicate bidirectionally whereby the notification module can communicate with the detection unit or the control unit bidirectionally via the relay unit.

When the second communication unit is provided in the control unit, the relay unit is provided in or near a headquarter. When a facility in which the notification system of the present invention is provided is a hospital, the headquarter is a nurse's station, for example. On the other hand, when the second communication unit is provided in the detection unit, the relay unit is provided near the detection unit (that is, in or near the room where the operator is present). When the facility in which the notification system of the present invention is provided is a hospital, the room is a sickroom of the patient.

In the notification system of the present invention, when it is determined that the predetermined event has occurred, the control unit transmits the second signal to the relay unit via the second communication unit and the first communication unit.

In the notification system of the present invention, the first communication unit and the second communication unit are wireless communication unit.

In the notification system of the present invention, the detection unit is an imaging unit configured to be able to generate videos. The control unit includes a third display module configured to be able to display videos. The control unit displays a video imaged by the imaging unit in a predetermined period including the time when the determination was made or the input operation was executed on the third display module when it is determined that the predetermined event has occurred or the input operation was performed.

In the notification system of the present invention, the control unit determines the occurrence of the predetermined event based on a change in a luminance value of a pixel that forms the video in response to a predetermined movement of the operator.

The notification system of the present invention includes a third communication unit configured to be able to communicate with at least one mobile communication terminal. When it is determined that the predetermined event has occurred, the control unit transmits information based on the result of the determination to the mobile communication terminal via the third communication unit. The relay unit notifies the mobile communication terminal of information indicating that the third signal has been transmitted via the first communication unit.

The control unit can specify the operator based on the movement information of the operator generated by the detection unit and the predetermined correlation information between the detection unit and the operator. The control unit determines a notification destination corresponding to the operator by this specification. Further, the control unit may control so that the information on the determined notification destination is displayed on the display module via the relay unit.

In the notification system of the present invention, the mobile communication terminal includes a fourth display module configured to be able to display videos. The control unit transmits a video corresponding to the predetermined event occurred to the mobile communication terminal via the second communication unit or the third communication unit together with the information indicating the execution of the determination. The mobile communication terminal displays the video on the fourth display module.

In the notification system of the present invention, the notification unit includes a switch which is the operating module and a call module which is the notification module. The notification unit is a nurse call system in which the call module operates when the operator operates the switch.

### Advantageous Effects of Invention

According to the present invention, it is possible to appropriately notify a healthcare provider of a change in a patient's state in a sickroom. For example, when a change occurs to a patient and a notification is output to a headquarter, it is possible to understand the patient's state in a period before and after the occurrence time with a predetermined operation. Alternatively, it is possible to output a notification to a headquarter when a change occurs to a patient or the like regardless of the presence of a call operation while using an existing notification system.

### BRIEF DESCRIPTION OF DRAWINGS

FIG. 1 is a functional block diagram illustrating an example of a nurse call system according to a first embodiment.

FIG. 2 is a functional block diagram illustrating an example of the nurse call system according to the first embodiment.

FIG. 3 is a table illustrating an example of correlation between the type of event movement information and an event threshold.

FIG. 4 is a table illustrating an example of correlation among the presence of occurrence of an event, the type of the occurred event, and an applied storage priority value.

FIG. 5 is a functional block diagram illustrating an example of an overall configuration of the nurse call system according to the first embodiment.

FIG. 6 is a table illustrating an example of an imaging unit and an operating module corresponding to a sickbed number.

FIG. 7 is a block diagram illustrating an example of a hardware configuration of the nurse call system according to the first embodiment.

FIG. 8 is a flowchart illustrating an example of a use form of the nurse call system according to the first embodiment.

FIG. 9 is a functional block diagram illustrating an example of a nurse call system according to a second embodiment.

FIG. 10 is a functional block diagram illustrating an example of the nurse call system according to the second embodiment.

FIG. 11 is a functional block diagram illustrating an example of an overall configuration of the nurse call system according to the second embodiment.

FIG. 12 is a table illustrating an example of an imaging unit, an operating module, and a mobile communication terminal corresponding to a sickbed number.

FIG. 13 is a block diagram illustrating an example of a hardware configuration of the nurse call system according to the second embodiment.

FIG. 14 is a flowchart illustrating an example of a use form of the nurse call system according to the second embodiment.

FIG. 15 is a flowchart illustrating a use form of a nurse call system according to Example.

FIG. 16 is a functional block diagram illustrating an example of a nurse call system according to a third embodiment.

FIG. 17 is a functional block diagram illustrating an example of the nurse call system according to the third embodiment.

FIG. 18 is a functional block diagram illustrating an example of an overall configuration of the nurse call system according to the third embodiment.

FIG. 19 is a table illustrating an example of a microwave Doppler sensor, an operating module, and a mobile communication terminal corresponding to a sickbed number.

FIG. 20 is a block diagram illustrating an example of a hardware configuration of the nurse call system according to the third embodiment.

FIG. 21 is a flowchart illustrating an example of a use form of the nurse call system according to the third embodiment.

FIG. 22 is a functional block diagram illustrating an example of a nurse call system according to a fourth embodiment.

FIG. 23 is a functional block diagram illustrating an example of the nurse call system according to the fourth embodiment.

FIG. 24 is a functional block diagram illustrating an example of a communication form of a control unit and a relay unit.

FIG. 25 is a functional block diagram illustrating an example of a communication form of a control unit and a relay unit.

FIG. 26 is a functional block diagram illustrating an example of an overall configuration of the nurse call system according to the fourth embodiment.

FIG. 27 is a table illustrating an example of an imaging unit, a relay unit, and an operating module corresponding to a sickbed number.

FIG. 28 is a block diagram illustrating an example of a hardware configuration of the nurse call system according to the fourth embodiment.

FIG. 29 is a flowchart illustrating an example of a use form of the nurse call system according to the fourth embodiment.

FIG. 30 is a functional block diagram illustrating an example of a nurse call system according to a fifth embodiment.

FIG. 31 is a functional block diagram illustrating an example of a communication form of a control unit and a relay unit.

FIG. 32 is a functional block diagram illustrating an example of a communication form of a relay unit and a notification unit.

FIG. 33 is a functional block diagram illustrating an example of an overall configuration of the nurse call system according to the fifth embodiment.

FIG. 34 is a block diagram illustrating an example of a hardware configuration of the nurse call system according to the fifth embodiment.

FIG. 35 is a functional block diagram illustrating an example of a nurse call system according to a sixth embodiment.

FIG. 36 is a functional block diagram illustrating an example of the nurse call system according to the sixth embodiment.

FIG. 37 is a functional block diagram illustrating an example of an overall configuration of the nurse call system according to the sixth embodiment.

FIG. 38 is a table illustrating an example of an imaging unit, an operating module, and a mobile communication terminal corresponding to a sickbed number.

FIG. 39 is a block diagram illustrating an example of a hardware configuration of the nurse call system according to the sixth embodiment.

FIG. 40 is a flowchart illustrating an example of a use form of the nurse call system according to the sixth embodiment.

FIG. 41 is a functional block diagram illustrating a conventional nurse call system.

### DESCRIPTION OF EMBODIMENTS

Hereinafter, embodiments of the present invention will be described with reference to the drawings. Constituent elements having the same configuration throughout the respective drawings will be denoted by the same reference numerals.

### First Embodiment

### Nurse Call System

Hereinafter, a configuration of a nurse call system according to this embodiment will be described with reference to the drawings. The description will be given in the following order: Functional configuration of nurse call system; Overall configuration of nurse call system; and Hardware configuration for realizing functional configuration.

[Functional Configuration of Nurse Call System]

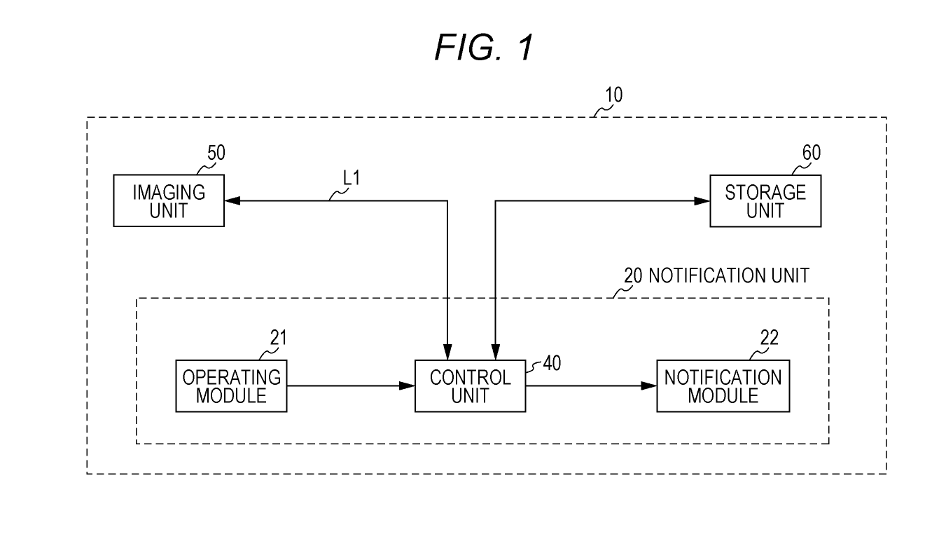

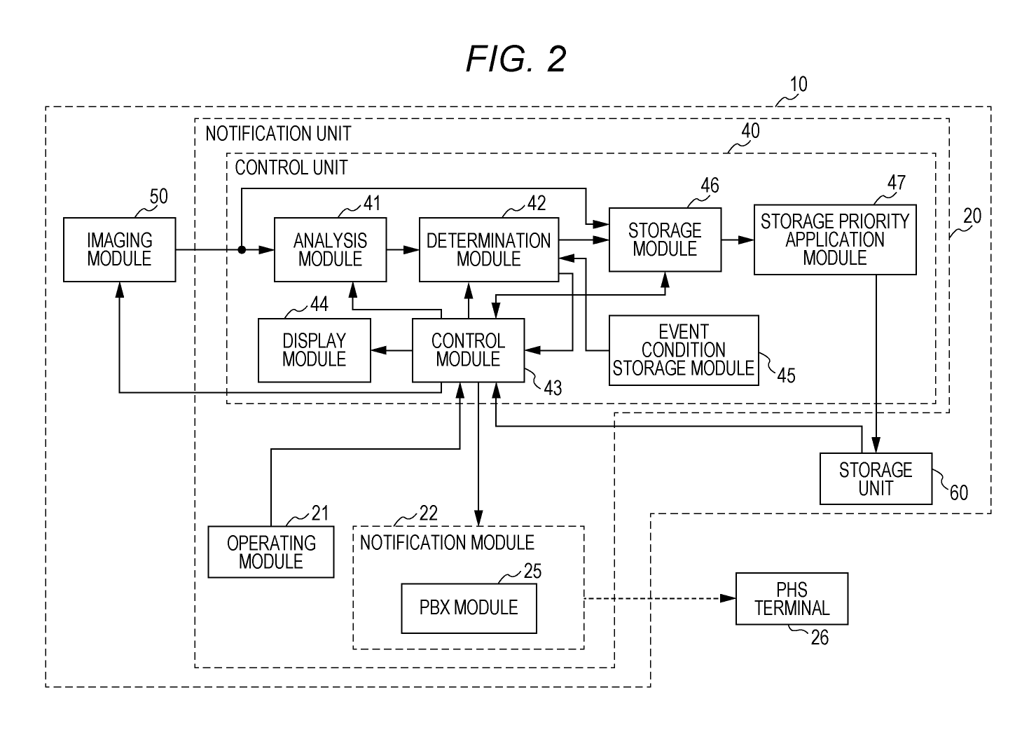

FIGS. 1 and 2 are functional block diagrams illustrating an example of a nurse call system according to this embodiment. FIG. 2 illustrates an example of a detailed configuration of a nurse call system 10 illustrated in FIG. 1. In the following description, a case in which a subject is a “patient” will be described.

[Nurse Call System 10]

As illustrated in FIG. 1, the nurse call system 10 includes an imaging unit 50, a notification unit 20, and a storage unit 60. The imaging unit 50 generates video information of a patient and outputs the video information to the notification unit 20. The video information includes movement information of the patient. The notification unit 20 includes an operating module 21, a notification module 22, and a control unit 40. The control unit 40 analyzes the video information received from the imaging unit 50. The control unit 40 further determines the presence of the occurrence of a predetermined event based on the analysis result. When it is determined that a predetermined event has occurred, the notification module 22 notifies an external device of the occurrence of the event. Moreover, the control unit 40 receives the determination result and outputs video information (hereinafter sometimes referred to as “detection video information”) including the patient movement information, corresponding to the occurred event, to the storage unit 60. Here, the “patient movement information” includes information that the patient has no movement as well as the information on the patient's movement. Examples of the “information that the patient has no movement” include an immovable state of the patient, a state in which the patient moves out of a detection range and is not in an imaging range which is the detection range, and the like.

(Imaging Unit 50)

The imaging unit 50 functions as a first sensor that generates detection video information. The detection video information is continuously generated for a predetermined period, for example.

The imaging unit 50 is provided so as to be able to generate a video (hereinafter sometimes referred to as a “detection video”) including the movement of a patient with a change over time among videos. The imaging unit 50 can be configured by selecting appropriately from those described as examples of an imaging device. The imaging unit 50 is provided at such a position that the bed of a patient is included in an imaging area thereof, for example.

The imaging unit 50 is configured so that the detection video information can be output from the imaging unit 50 to the control unit 40 by being connected to the control unit 40 via a communication line L1. The communication line L1 may be an arbitrary element as long as it includes a configuration that realizes input and output between the imaging unit 50 and the control unit 40 by communication. The communication line L1 may be configured by a wireless element, a cable element, or a combination thereof, for example. Moreover, the communication line L1 preferably includes a configuration that can periodically check establishment of communication.

When the communication line L1 is a cable LAN path, a router having the function of PoE (Power over Ethernet (registered trademark)) is preferably provided in the LAN path. In this case, the use of the PoE function enables power to be supplied from the router to the imaging unit 50 via the cable LAN.

(Storage Unit 60)

The storage unit 60 stores the detection video information generated by the imaging unit 50. The imaging unit 50 is connected to the storage unit 60 via the control unit 40 whereby the detection video information subjected to predetermined processing by the control unit 40 is input to the storage unit 60. The detection video information is detection video information to which a storage priority value described later is applied, for example. Moreover, the detection video information is detection video information which has been subjected to privacy processing, for example. Examples of the privacy processing include mosaic processing on the face of a patient, processing of painting the silhouette of a person in single color.

The storage unit 60 may have a function of removing or moving the stored detection video information according to a predetermined condition. Examples of the predetermined condition include the elapse of a predetermined period from the time when it was stored in the storage unit 60, an available volume of the storage unit 60 being lower than a predetermined value, and the like. The removal or the movement is executed on a plurality of items of stored detection video information in ascending order of the storage priority values applied thereto. As a specific example, a storage determination module (not illustrated) included in the storage unit 60 determines whether the detection video information of which the storage period has elapsed a predetermined period from the time when it was stored in the storage unit 60 will be removed or moved. This determination is made based on the storage priority value applied to the detection video information. In this determination, when the storage priority value is smaller than a predetermined threshold, the detection video information is removed or moved.

The detection video information of which the movement is determined to be needed may be moved, for example, to a cloud server (not illustrated) or the like connected to the storage unit 60 via a network (not illustrated). Moreover, the storage unit 60 may not always need to have the function of removing or moving the detection video information, and for example, a determination module 42 or a control module 43 may remove or move the detection video information stored in the storage unit 60.

The storage unit 60 may be a server provided via a network N. In communication between the servers, a communication system compliant with DICOM may be used for communication of videos or the like. In this case, the storage unit 60 forms a picture archive communication system (PACS) in which videos are stored, a hospital information system (HIS) in which patient information, hospital information, and the like are stored, and the like.

(Notification Unit 20)

The notification unit 20 is a nurse call device that outputs a notification to an external device based on the input of a predetermined operation by the patient.

The operating module 21 and the notification module 22 are provided so that a signal can be output from at least the operating module 21 to the notification module 22 via the control unit 40. This output is performed via communication of a transceiver module (not illustrated), for example. In this case, the signal transmitted (output) from the operating module 21 to the notification module 22 is received (input) to the notification module 22 via the control unit 40.

The notification module 22 is provide at a position separated from the operating module 21. Specifically, the operating module 21 is provided in a sickroom and the notification module 22 is provided in a nurse's station. For example, when a patient operates the operating module 21, at least a signal (hereinafter sometimes referred to as a “notification module activation signal”) instructing the control unit 40 to activate the notification module 22 is transmitted (output) from the operating module 21 in response to the input of the operation. The control unit 40 controls the notification module 22 in response to this signal whereby the notification module 22 is activated and a notification is output to an external device. In this case, although various items of information such as control information or identification information of the operating module 21 are output from the operating module 21 in addition to the notification module activation signal, the notification module activation signal may include these various items of information.

<<Operating Module 21>>

The operating module 21 may be an arbitrary element as long as it includes a configuration capable of outputting a predetermined signal to the control unit 40 based on the input of an operation. In this case, the predetermined signal includes a predetermined instruction on the control unit 40. The predetermined instruction is an instruction to activate the notification module 22 to output a notification to an external device, for example. That is, the predetermined signal includes a notification module activation signal. A conventional existing button or trigger-type nurse call extension module, a conventional existing nurse call button having a switch button, and the like can be appropriately selected and used as the operating module 21, for example.

<<Notification Module 22>>

The notification module 22 may be an arbitrary element as long as it is configured to output a notification to an external device in response to the notification module activation signal. Examples of the form of the notification output by the notification module 22 include those sensed by the five senses of a person, such as sound, light, or vibration.

Moreover, the notification unit 20 may include a PBX module 25. For example, the PBX module 25 is provided in the notification module 22 as illustrated in FIG. 2. The notification module 22 outputs a notification to an external device in response to a control signal from the control unit 40 and activates the PBX module 25. The PBX module 25 transmits a signal to a PHS terminal 26. The PHS terminal 26 having received this signal performs an operation of outputting a notification to an external device.

<<PBX Module 25>>

As long as the PBX module 25 can receive the signal from the control unit 40, the installation form and the connection form thereof are not particularly limited. Moreover, the PHS terminal 26 may be connected to the notification module 22 and may be connected to the notification unit 20.

<<Control Unit 40>>

The control unit 40 analyzes the detection video information received from the imaging unit 50. The control unit 40 determines the presence of the occurrence of a predetermined event based on the analysis result. When it is determined that a predetermined event has occurred, the control unit 40 outputs detection video information corresponding to the event to the storage unit 60.

As illustrated in FIG. 2, the control unit 40 includes an analysis module 41, a determination module 42, a control module 43, a display module 44, an event condition storage module 45, and a storage priority application module 47. Moreover, a storage module 46 in which the detection video information is temporarily stored is provided as necessary between the storage priority application module 47 and the storage unit 60.

<Analysis Module 41>

The analysis module 41 analyzes the detection video information received from the imaging unit 50 to specify movement information of the patient and outputs the movement information to the determination module 42. When the detection video information is continuously generated by the imaging unit 50, the analysis module 41 continuously receives the detection video information, analyzes the detection video information on a real-time basis, and outputs the analysis result to the determination module 42.

The analysis module 41 analyzes the detection video information based on a predetermined algorithm (method). In this way, the movement information of the patient is specified from the detection video information. According to an example of a video analysis algorithm, edges are extracted from the detection video information, and the shapes of the edges, a moving object, and the like are analyzed to detect that the object is a person. When this algorithm is used, video analysis is performed in the following manner, for example.

First, the analysis module 41 applies a predetermined filter to the detection video information received from the imaging unit 50 to extract edges. Examples of the filter include a filter that enables a boundary of an object to be detected based on the luminance value of a pixel, a filter that enables a moving object to be detected based on a change in the luminance value of a pixel.

Subsequently, the analysis module 41 performs a matching process based on the size, the shape, the movement, or the like of a portion surrounded by the extracted edges. With this matching process, a portion of the detection video, surrounded by the edges is selected. This matching process is performed by comparing matching information stored in advance in a matching information storage module (not illustrated) with information on the portion surrounded by the edges. The matching information includes information such as the size of a person, the shape of a person, or the movement of a person, for example. With this process, the portion surrounded by the edges is specified as a person. Further, the movement information of the portion surrounded by the edges is specified as the patient movement information and is output to the determination module 42.

According to another example of the video analysis algorithm, a filtering process is performed on the detection video information generated by a thermography camera to specify an area of the detection video, corresponding to a specific temperature distribution. In this example, a matching process is similarly performed on the specified area.

The video analysis is not limited to that based on the algorithms mentioned above. For example, a conventional existing algorithm can be appropriately selected and used for the video analysis. Further, a plurality of alignments may be combined and used for the video analysis. When the determination module 42 determines that a predetermined event has occurred, the determination module 42 outputs information (hereinafter sometimes referred to as “event occurrence information”) indicating the occurrence of the event to the control module 43.

<Determination Module 42>

The determination module 42 determines the presence of the occurrence of a predetermined event based on the analysis result received from the analysis module 41. The determination module 42 determines whether the patient movement information received from the analysis module 41 is identical to predetermined movement information (predetermined event; hereinafter sometimes referred to as “event movement information”).

The “predetermined event” means a specific movement of a patient. Examples of the specific movement include a “movement that a patient tries to get up from a bed,” a “movement that a patient tries to leave a bed,” a “movement that a patient has tumbled,” and the like. When the determination module 42 determines that the “predetermined event has occurred,” the determination module 42 outputs the determination result to at least the control module 43. In this case, the “identical” includes “substantially identical” as well as “perfectly identical.” Examples of the “substantially identical” include a case in which a portion of a series of movements of a patient is identical to the predetermined movement information. Moreover, the analysis module 41 may specify a body axis of the patient from the matching process, for example. The determination module 42 may determine that the predetermined event has occurred when the movement of the specified body axis is identical to movement information of the body axis included in the event movement information.

The determination module 42 is not limited to determine the presence of the occurrence of the predetermined event based on the identicalness of the movement information of the patient only. For example, when the patient is absent in a sickroom in a specific period, it is determined that the movement information of the patient is “Absent.” However, in this state, the determination module 42 may determine that the predetermined event has occurred. The predetermined event is “Wandering,” for example. As an example, when the determination time of the occurrence of the event is “night (after lights-out)” and a patient is absent from a sickroom in a specific period, the determination module 42 determines that an event of “Wandering” has occurred.

Moreover, the determination module 42 specifies detection video information (hereinafter sometimes referred to as “event detection video information”) including a video indicating the occurrence of the predetermined event from the detection video information stored in the storage unit 60 and sequentially outputs the specified detection video information to the storage unit 60.

Specifically, the event detection video information is detection video information generated by the imaging unit 50 in a predetermined period including the occurrence time (hereinafter sometimes referred to as an event occurrence time) of the predetermined event. For example, the starting point of the predetermined period is the time occurring a first predetermined period earlier than the event occurrence time and the ending point thereof is the time occurring a second predetermined period after the event occurrence time. Moreover, the event detection video information may be generated in the above-described manner using the time at which the operation of the operating module 21 is input to the control module 43 as the event occurrence time, for example. When the determination of the determination module 42 is performed on a real-time basis, the “event occurrence time” is the time at which it is determined that the predetermined event has occurred.

<Event Condition Storage Module 45>

Event condition information is stored in advance in the event condition storage module 45. The “event condition information” is information used for determination of the occurrence of the predetermined event. The “event movement information” is included in the event condition information. Moreover, an “event threshold” is included in the “event condition information” as a threshold of the number of times of the identicalness determination. In this case, the determination module 42 determines that “the predetermined event has occurred” when the number of times of the identicalness determination reaches a predetermined number. The event condition storage module 45 is connected to the determination module 42. In this way, when the above-described determination is performed, the “event condition information” is output to the determination module 42.

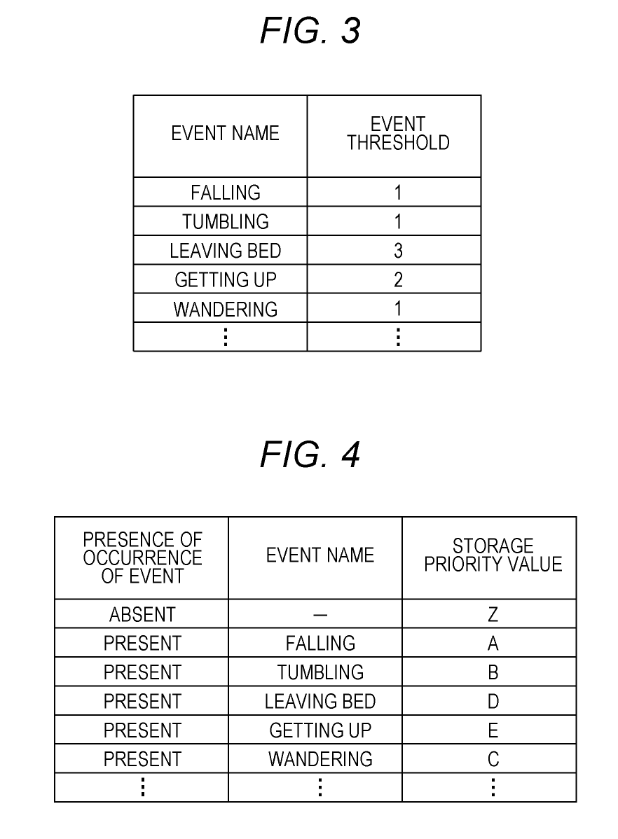

The “event threshold value” is set according to the type of the event movement information, for example. FIG. 3 is a table illustrating an example of the correlation between the type of the event movement information and the “event threshold.”

As illustrated in FIG. 3, an event name is set as the type of the predetermined event, and for example, “Falling,” “Tumbling,” “Wandering,” “Leaving Bed,” “Getting Up,” and the like are set as the event name. In such a correlation as illustrated in FIG. 3, an event threshold is set for each event type. For example, the event threshold is set to “1” for the event type such as “Falling,” “Tumbling,” and “Wandering.” That is, when the number of determinations associated with the identicalness between the patient movement information received from the analysis module 41 and the event movement information corresponding to these types is “1,” it is determined that the predetermined event has occurred. This is because the movements of a patient associated with these types of events are highly likely to lead to a serious accident which can change the life of the patient. Thus, these types of events have a high level of urgency to response among the predetermined events and require an immediate notification of the notification module 22.

<Control Module 43>

The control module 43 controls respective constituent elements of the nurse call system 10 in response to the event occurrence information from the determination module 42. The control module 43 controls the display module 44 and the notification module 22, for example, among the respective constituent elements. In this case, a live video based on the detection video information output from the imaging unit 50 or a detection video based on the event detection video information is displayed on the display module 44, for example. Moreover, the notification module 22 emits a notification sound based on the notification module activation signal output from the control module 43.

The control module 43 may control the respective constituent elements of the nurse call system 10 in response to the notification module activation signal output from the operating module 21. The control module 43 controls the display module 44 in the above-described manner, for example, among the respective constituent elements.

The control module 43 can appropriately control the imaging unit 50, the notification module 22, and the respective constituent elements (the analysis module 41, the determination module 42, the storage module 46, and the like) of the control unit 40 as necessary.

When a notification module activation signal is output to the notification module 22, the control module 43 may store the transmission history of the notification module activation signal in the storage unit 60 or the like. In this case, the identification information of the imaging unit 50, the type of the predetermined event, the event occurrence date and time, and the like when it is determined that the predetermined event has occurred are stored in association with the transmission history.

When the output of the notification module activation signal is based on the event occurrence information, the identification information of the operating module 21, the identification information of the imaging unit 50, the type of the predetermined event, the operation input data and time, the event occurrence date and time, and the like are stored in the storage unit 60 or the like in association with the transmission history, for example. Moreover, in this case, the occurrence information of the predetermined event may be selectively stored.

<Display Module 44>

The display module 44 is configured to be able to display a live video based on the detection video information output from the imaging unit 50 or the detection video based on the event detection video information. The display module 44 may be an arbitrary element as long as it has a configuration capable of displaying videos. The display module 44 preferably has a configuration capable of displaying color videos, for example. The display module 44 corresponds to a “first display module” of the present invention.

The display module 44 may have the function of the notification module 22 by displaying notification information (for example, character information). Moreover, the display module 44 may be configured to emit a notification sound via a sound playback module (not illustrated) as well as displaying the information. In this case, the connection of the notification module 22 to the control unit 40 may be omitted. That is, the notification unit 20 may be configured to eliminate the notification module 22. Moreover, when the notification of the nurse call system 10 is limited to a visual notification, the notification module 22 may be omitted.

The control unit 40 includes a storage module 46 and a storage priority application module 47 for performing a predetermined process on the detection video information output from the determination module 42 to the storage unit 60. The storage module 46 is a temporary storage module in which the detection video information continuously generated by the imaging unit 50 is sequentially stored and is provided as necessary. Moreover, the storage priority application module 47 applies a storage priority value to the event detection video information among the items of input detection video information, for example.

<Storage Module 46>

The detection video information generated by the imaging unit 50 is temporarily stored in the storage module 46. The control module 43 can select the detection video information based on the event detection information. A specific example of how detection video information is selected will be described below.

The detection video information output from the imaging unit 50 is sequentially stored in the storage module 46. In this case, the presence of the occurrence of the predetermined event is determined by the determination module 42 simultaneously with the storage. In this determination, when it is determined that the predetermined event has occurred, event occurrence information is output to the control module 43. The control module 43 removes the detection video information other than the event detection video information among the items of detection video information stored in the storage module 46 in response to the event occurrence information. This removal is executed when it is determined that an event has not occurred after the elapse of a first predetermined period from the generation time of the detection video information. Moreover, the removal is resumed when a second predetermined period elapses from the determination time of the occurrence of the event and a new event has not occurred. In this way, the event detection video information is selectively left in the storage module 46. The event detection video information is stored in the storage unit 60 after the storage priority value is applied thereto by the storage priority application module 47, for example.

As described above, the storage module 46 functions as a temporary storage module when the event detection video information is specified from the detection video information and the storage priority value is applied to the detection video information. Since the storage module 46 is provided in the control unit 40, an information readout speed of the storage module 46 may be faster than the storage unit 60. Thus, the event detection video information which is highly likely to be reproduced may be stored in the storage module 46.

In this case, the video information stored in the storage module 46 is event detection video information to which a high storage priority value is applied. The “high storage priority value” corresponding to the video information is the storage priority values “A” and “B” illustrated in the table of FIG. 4 described later. The event names corresponding to these storage priority values are specified as “Tumbling” and “Falling” from the table of FIG. 4. That is, the event detection video information indicating the occurrence of “Tumbling” and “Falling” is left in the storage module 46 by being copied to the storage unit 60. The event detection video information indicating the occurrence of the other events is removed from the storage module 46 by being moved to the storage unit 60. In this way, the event detection video information to which a high storage priority value is applied is stored by being selectively left in the storage module 46. Thus, even when an event having a high level of urgency to response occurs to a patient, a healthcare provider can check an event detection video corresponding to the event on the display module 44 or the like by reading the event detection video from the storage module 46 without accessing the storage unit 60. Moreover, since the available volume of the storage module 46 is limited, the event detection video information is removed or moved based on the predetermined conditions similarly to the storage unit 60. In this case, the destination of the event detection video information is the storage unit 60, for example.

<Storage Priority Application Module 47>

The storage priority application module 47 applies a storage priority value to the detection video information including the event detection video information based on the event occurrence information and the storage condition information received from the determination module 42. The detection video information may be received directly from the imaging unit 50 and may be received via the storage module 46. In this case, it is assumed that all items of the detection video information generated by the imaging unit 50 are stored in the storage unit 60.

Here, the “storage priority value” indicates the degree of priority of storage of the detection video information stored in the storage unit 60. In other words, the storage priority value indicates the degree of priority of removal or movement, and the detection video information is removed or moved preferentially from the detection video information of which the applied storage priority value is lower. Moreover, the items of detection video information having the same storage priority value are removed or moved in ascending order of the storage date and time, for example. The degree is determined according to the importance of the detection video information (that is, the degree of need to store), for example. The degree of need to store is determined depending on “whether the event movement information is included in the detection video information,” “the type of an event occurred,” and the like, for example. Although the storage priority value is applied to the detection video information, the storage priority value may be applied to the event detection video information only.

The “storage condition information” indicates the correlation between the event occurrence information and the storage priority value. The event occurrence information includes information indicating the presence of the occurrence of the event and information on the type of an event occurred. The “storage condition information” is stored in advance in the storage module 46, for example.

FIG. 4 is a table illustrating an example of the correlation among the “presence of occurrence of event,” the “type of occurred event,” and the applied storage priority value.

In the correlation illustrated in FIG. 4, the storage priority value is set to a case of no event occurred. Moreover, the same event name as illustrated in FIG. 3 is set as the type of the predetermined event for a case where an event has occurred. In the correlation, the storage priority value is set for each event type. The storage priority values are represented by A to Z. Among the storage priority values in the correlation, A has the highest storage priority value, the storage priority values decrease in the alphabetical order from A, and Z has the lowest storage priority value.

That is, the lowest storage priority value “Z” is correlated with the case of “No event occurred.” On the other hand, when events have occurred and the event types correspond to “Falling” and “Tumbling,” high storage priority values “A” and “B” are correlated with such cases. That is, the event detection video information corresponding to the event type of “Falling” and “Tumbling” has a high degree of need to store.

The “high degree of need to store” means that the event detection video information is highly likely to be used later, for example. For example, when a subject patient is injured by tumbling, the event detection video information at the time of tumbling may be stored and protected so that the information is used as a guideline for treatment of the injury. Moreover, the process of the tumbling may be extracted from the event detection video information and be stored for each patient as a history so that the history can be used later. Further, when the tumbling causes a dispute as a medical accident, by protecting the storage of the event detection video information, the event detection video information can be used as an evidence video in the place of dispute.

On the other hand, in the correlation, a storage priority value C is correlated with the event type of “Wandering.” This is because the “Wandering” state requires a higher level of urgency to response than the “Getting Up” and “Leaving Bed” states and it is determined that the need to leave the same as a history is high. Moreover, when an accident occurs during “Wandering,” the corresponding event detection video information can be used in later inspection. Further, when a plurality of events occurs simultaneously, a highest value among the storage priority values corresponding to these events is applied to the detection video information thereof, for example.

The storage condition information may not include a storage priority value corresponding to the case of “No event occurred.” Based on this correlation information, the storage priority application module 47 does not apply a storage priority value to the detection video information other than the event detection video information. In this case, the detection video information corresponding to the storage priority value “Z” illustrated in FIG. 4 is detection video information to which the storage priority value is not applied. Moreover, when the detection video information other than the event detection video information is selectively removed by the control module 43, the storage condition information may not include the storage priority value corresponding to the case of “No event occurred.”

In this way, a high storage priority value is applied to the event detection video information that is to be used later, the event detection video information that is to be left as a history, and the like, whereby these items of event video information are protected in the storage unit 60.

[Overall Configuration of Nurse Call System 10]

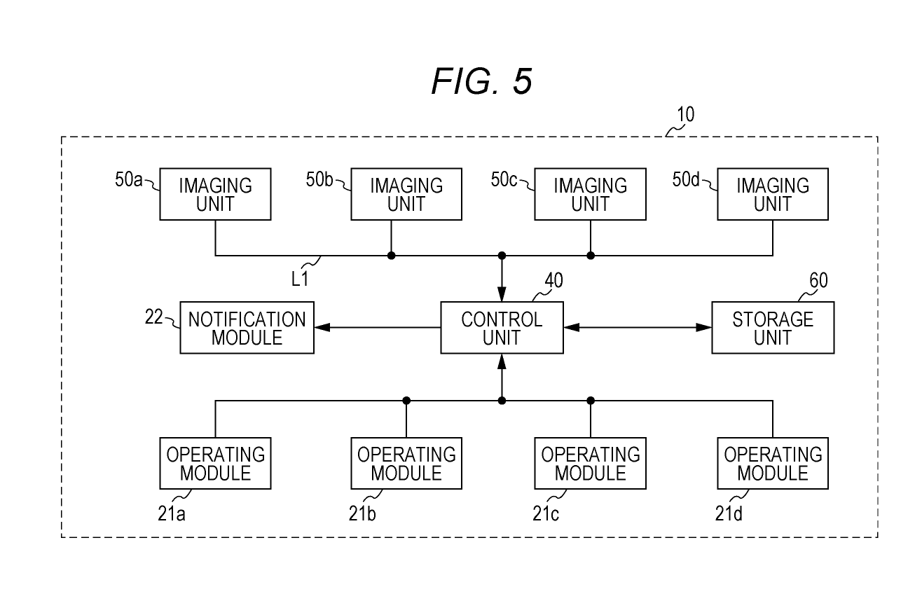

Next, an overall configuration of the nurse call system 10 according to this embodiment will be described. FIG. 5 is a block diagram illustrating an example of an overall configuration of the nurse call system 10 according to this embodiment. A number of patients are hospitalized in a hospital in which the nurse call system. 10 is used. Thus, the nurse call system 10 includes the same number of operating modules 21 as the number of sickbeds and includes the same number of imaging unit 50 as the number of sickbeds.

As illustrated in FIG. 5, the nurse call system 10 is configured such that a plurality of imaging unit 50 is connected to the control unit 40 so that the imaging unit 50 can output and input information to and from the control unit 40. The plurality of imaging unit 50 (50a to 50d) are connected by a communication line L1 so as to be able to transmit and receive (output and input) information to and from the control unit 40. Further, the storage unit 60 and the notification module 22 are connected to the control unit 40. The storage unit 60 is connected so as to be able to transmit and receive information to and from the control unit 40. The notification module 22 is connected so that at least information can be transmitted from the control unit 40 to the notification module 22. Although the nurse call system 10 illustrated in the drawing illustrates a case in which the number of sickbeds is four, the number of sickbeds is not limited thereto but can be set appropriately (the same herein below).

The correlation between the nurse call system 10 illustrated in FIG. 5 and the nurse call system 10 illustrated in FIG. 1 will be described by way of an example of the imaging unit 50a and the operating module 21a illustrated in FIG. 5. In the nurse call system 10 illustrated in FIG. 5, the imaging unit 50a and the operating module 21a which are correlated are connected so as to be able to transmit and receive information to and from the control unit 40 and the notification module 22, whereby the same configuration as the nurse call system 10 illustrated in FIG. 1 is obtained. Moreover, the same correlation is similarly applied to the imaging unit 50b to 50d and the operating modules 21b to 21d. That is, the overall configuration of the nurse call system 10 is configured such that the plurality of sets of imaging unit and operating modules which are correlated are connected so as to be able to transmit and receive information to and from the control unit 40 and the notification module 22.

Correlation information among a sickbed number, the imaging unit 50, and the operating module 21, for example, is stored in advance in the storage unit 60 or the storage module 46 (hereinafter sometimes referred to as the “storage unit 60 or the like”). This correlation information is represented by a correlation between the unique IDs (hereinafter sometimes referred to as “UIDs”) which are identifiers applied to the sickbed number, the imaging unit 50, and the operating module 21, for example.

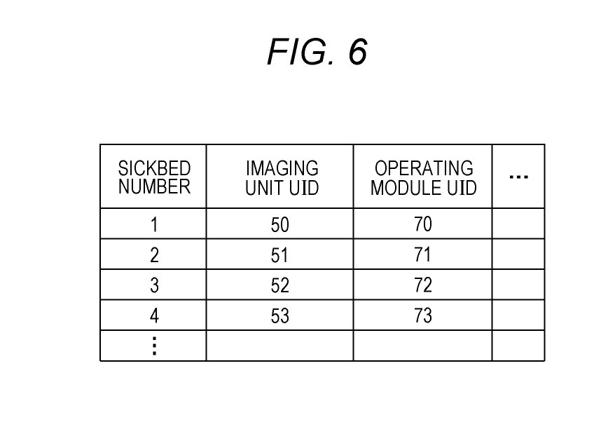

FIG. 6 is a table illustrating an example of the imaging unit 50 and the operating module 21 corresponding to the sickbed number. As illustrated in FIG. 6, this table illustrates the correlation among a sickbed number (hereinafter sometimes referred to as a “sickbed UID”) applied to each sickbed, an imaging unit UID (hereinafter sometimes referred to simply as “imaging unit UID”) applied to each imaging unit 50, and an operating module UID (hereinafter sometimes referred to simply as “operating module UID”) applied to each operating module 21.

The determination module 42 outputs “event occurrence information that specifies the sickbed” to the control module 43 and the storage unit 60 or the like based on the correlation. The “event occurrence information that specifies the sickbed” includes “information (sickbed number or the like) for specifying the sickbed” and “event detection information that specifies the sickbed.” The control module 43 outputs the “information for specifying the sickbed” and the like to the notification module 22, the display module 44, and the like. Moreover, the “event detection information that specifies the sickbed” is output to the storage unit 60 or the like.

The notification module 22 outputs a notification (an audio notification such as a sickbed number) that specifies the sickbed in response to the “information for specifying the sickbed.” The display module 44 displays information (information such as a sickbed number) that specifies the sickbed in response to the “information for specifying the sickbed.” With the notification and the information, a healthcare provider can recognize the sickbed in which an event has occurred. The storage unit 60 stores the event detection information in association with the sickbed information in response to the “event detection information that specifies the sickbed.”

With reference to the table in FIG. 6, an example in which the determination module 42 outputs “the event occurrence information that specifies the sickbed” will be described.

First, as a first example, a case in which the detection video information is generated by the imaging unit 50a will be described. In this case, the imaging unit UID “50” is associated with the detection video information. Upon receiving the detection video information, the determination module 42 specifies the sickbed number “1” corresponding to the detection video information based on the associated imaging unit UID “50” and the correlation information. Further, the determination module 42 outputs information including the information of the sickbed number “1.” The determination module 42 outputs a notification module activation signal (notification information that specifies the sickbed) including the information of sickbed number “1,” for example, to the notification module 22. The notification module 22 outputs a notification including the sound of “sickbed number 1” in response to the information. The determination module 42 outputs the event detection video information corresponding to the event to the display module 44 in association with the information of sickbed number “1,” for example. The display module 44 displays the event detection video together with the character of sickbed number “1” in response to the information. The determination module 42 outputs the event detection video information (the event detection information that specifies the sickbed) corresponding to the event to the storage unit 60 in association with the sickbed number “1.” The storage unit 60 stores the event detection video together with the character information of sickbed number “1” in response to the information.

As a second example, a case in which a notification module activation signal is output from the operating module 21a will be described. In this case, the operating module UID “70” is associated with the signal. Upon receiving the signal, the determination module 42 specifies the sickbed number “1” and the imaging unit UID “50” corresponding to the signal based on the operating module UID “70” and the correlation information. Further, the determination module 42 generates event detection video information from the detection video information generated by the imaging unit 50a corresponding to the imaging unit UID “50.” Further, the determination module 42 outputs information including the information of sickbed number “1.” This information is output similarly to the first example.

An identifier correlated with the sickbed number is not limited to that described above but may be set arbitrarily. For example, an identifier (a patient UID, a nurse UID, an assistant UID, a physician UID, or the like) applied to a patient, a healthcare provider, or the like may be used for setting the correlation information. The same is true for the correlation information illustrated below.

When a patient UID is correlated with a sickbed number, the event threshold illustrated in FIG. 3 may be changed based on the patient UID. That is, the determination module 42 specifies an event which is likely to occur to a specific patient and increases or decreases the event threshold from a standard value based on the specification result.

Specifically, the determination module 42 specifies a sickbed in which the same event has occurred a number of times within a predetermined period from an event occurrence history. The determination module 42 specifies the patient UID from the sickbed number. When the event is “Wandering,” for example, the event threshold thereof is decreased.

Specifically, when the standard value of the event threshold of “Wandering” is “3,” the event threshold of “Wandering” is changed to “1.” This change is performed according to an event type, for example. Specifically, the event threshold is decreased only for an event such as “Tumbling” of which the degree of urgency to response is high (for example, the storage priority value is high), whereas the event threshold is increased only for an event such as “Getting Up” of which the degree of urgency to response to low (for example, the storage priority value is low).

The determination module 42 may generate new event condition information corresponding to a patient from the content (a combination of the types of occurred events) of an event occurrence history. In this case, the event condition information may be stored in the event condition storage module 45 together with the corresponding patient UID. The patient is a patient whose movement during occurrence of the event, for example, is characteristic. This “characteristic movement” is correlated with the patient as “event movement information” whereby the new event condition information is generated. In this manner, when new event condition information corresponding to a patient is generated and used for determination, the healthcare provider can more properly recognize the patient's state in each sickbed.

When the patient UID and the sickbed number are correlated, the determination module 42 may change the storage priority value illustrated in FIG. 5 based on the patient UID. As an example, when the event threshold is changed, for example, the determination module 42 specifies a sickbed in which the same event has occurred a number of times within a predetermined period from the event occurrence history. The determination module 42 specifies the patient UID from the sickbed number. The determination module 42 changes the storage priority value corresponding to the event based on the specification result. This change is performed according to an event type, for example. For example, the storage priority value is increased only for an event such as “Tumbling” of which the degree of urgency to response is high, whereas the storage priority value is decreased only for an event such as “Getting Up” of which the degree of urgency to response to low. The plurality of processes of specifying the sickbed or the patient may be performed by the control module 43 instead of the determination module 42.

[Hardware Configuration of Nurse Call System 10]

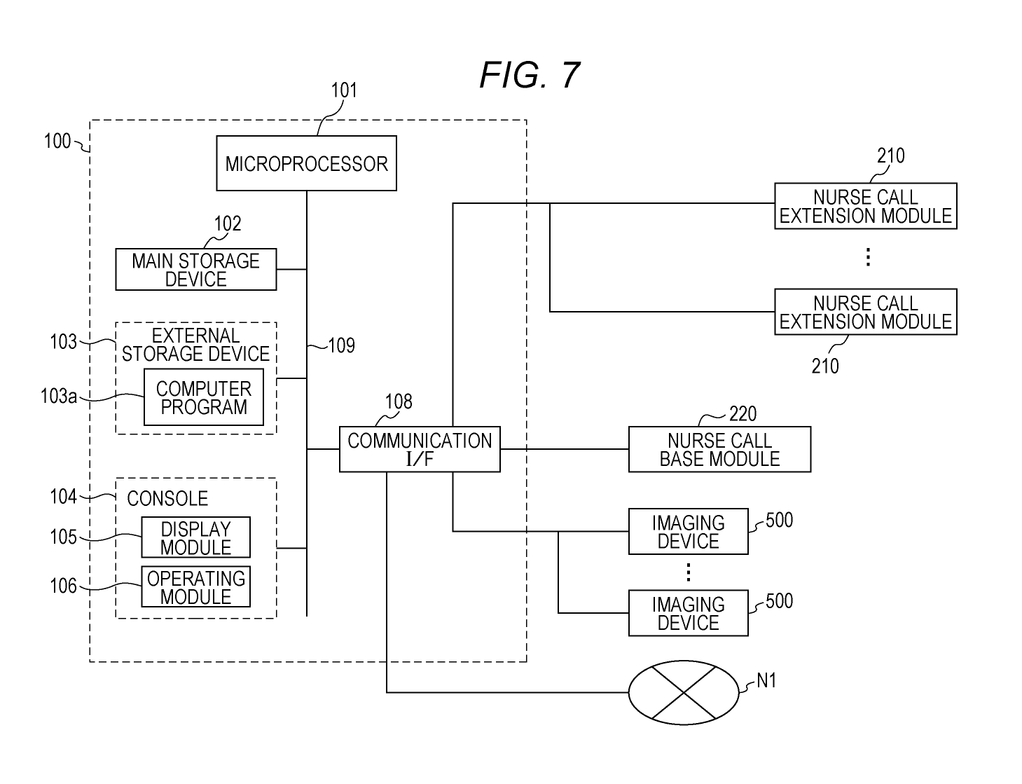

Next, a hardware configuration of the nurse call system 10 (mainly a computer 100) for realizing the above-described functions will be described with reference to FIG. 7. FIG. 7 is a block diagram illustrating an example of a hardware configuration of the nurse call system 10 according to this embodiment.

(Computer 100)

First, a computer having the function of the control unit 40 will be described. The computer 100 is configured to include a microprocessor 101, a main storage device 102, an external storage device 103, and a communication interface 108. Reference numeral 109 indicates a bus that connects these respective constituent elements. Hereinafter, these constituent elements of the computer 100 will be described.

The microprocessor 101 includes an arithmetic control device such as a CPU or a MPU. The operation of the control unit 40 is realized by the microprocessor 101. The main storage device 102 is configured to include a memory device such as a RAM. The external storage device 103 is configured to include a storage device such as a ROM or a hard disk drive. Information collected according to an instruction from the microprocessor 101 is temporarily stored in the main storage device 102 and the external storage device 103, for example. At least one of these storage devices corresponds to an example of the storage module 46 illustrated in FIG. 2. Moreover, at least one of these storage devices corresponds to an example of the event condition storage module 45 illustrated in FIG. 2.

A computer program 103a is stored in advance in the external storage device 103. The microprocessor 101 loads the computer program 103a on the main storage device 102 to cause the nurse call system 10 to perform the characteristic processes of this embodiment. The microprocessor 101 performs the processes to at least have the functions of the analysis module 41, the determination module 42, the control module 43, and the storage priority application module 47 illustrated in FIG. 2, for example.

The computer 100 has a console 104 as necessary. The console 104 is used by a healthcare provider, for example. This console includes a display module 105 and an operating module 106. The display module 105 is configured to include an arbitrary display device such as a liquid crystal (LCD) display, an organic EL (OLED) display, an electronic paper, or a CRT display. This display module 105 has the function of the display module 44 illustrated in FIG. 2, for example. Moreover, the operating module 106 is configured to include an arbitrary operating device or input device such as a keyboard, a mouse, a trackball, a joystick, or a console panel. Moreover, the display module 105 may include the operating module 106, and a touch panel display is an example thereof. The touch panel display is operated with the fingers of a healthcare provider, a touch pen, or the like. The operating module 106 has the function of the operating module 21 illustrated in FIG. 2, for example.

The communication interface (I/F) 108 is configured to include a network adapter (NIC) such as a LAN card and an arbitrary communication device such as a modem for connecting to the Internet.

A nurse call extension module 210, a nurse call base module 220, an imaging device 500, and an external network N1 are connected to the computer 100 via the communication interface 108. A communication interface (not illustrated) is also provided in these connected devices and has the same configuration as the communication interface 108, for example.

The imaging device 500 may be an arbitrary device as long as it has the function of the imaging unit 50 described above. The imaging device described above can be appropriately selected and used as the imaging device 500, for example. The detection video information generated by the imaging device 500 is transmitted to the main storage device 102, for example, via the communication interface 108, and is processed by the microprocessor 101. The imaging device 500 continuously generates the detection video information and sequentially transmits the same to the main storage device 102, for example. The microprocessor 101 sequentially processes the detection video information based on the above-described algorithm to make determination on the occurrence of a predetermined event on a real-time basis. Moreover, the detection video information is further transmitted to the external storage device 103 and the detection video information is continuously recorded on the external storage device 103.

The imaging device 500 is one form of the detection device and the detection device is not limited to this form. For example, the imaging device 500 may be configured by a moving object detection sensor so as to generate detection information. The detection information is processed by the computer 100 and the occurrence of a predetermined event is detected in a similar manner. The details of this form will be described in a third embodiment.

A communication interface (not illustrated) is provided in the nurse call extension module 210 and the nurse call base module 220. These communication interfaces are connected to the communication interface 108. Due to this, the computer 100 can receive (input) the signals from the nurse call extension module 210 via the communication interface 108 and transmit (output) a control signal to the nurse call base module 220.

The computer 100 is connected to the external network N1 by the communication interface 108. A file server (a NIS server, a FTP server, or the like), a picture server (a PACS server or the like), a mail server (a POPS server, a SMTP server, or the like), a WEB server (a HTTP server or the like), and the like are connected to the external network N1 so as to be able to transmit and receive (output and input) information. The computer 100 can output and input information to and from an external device by being connected to these servers as necessary.

[Use Form of Nurse Call System 10]

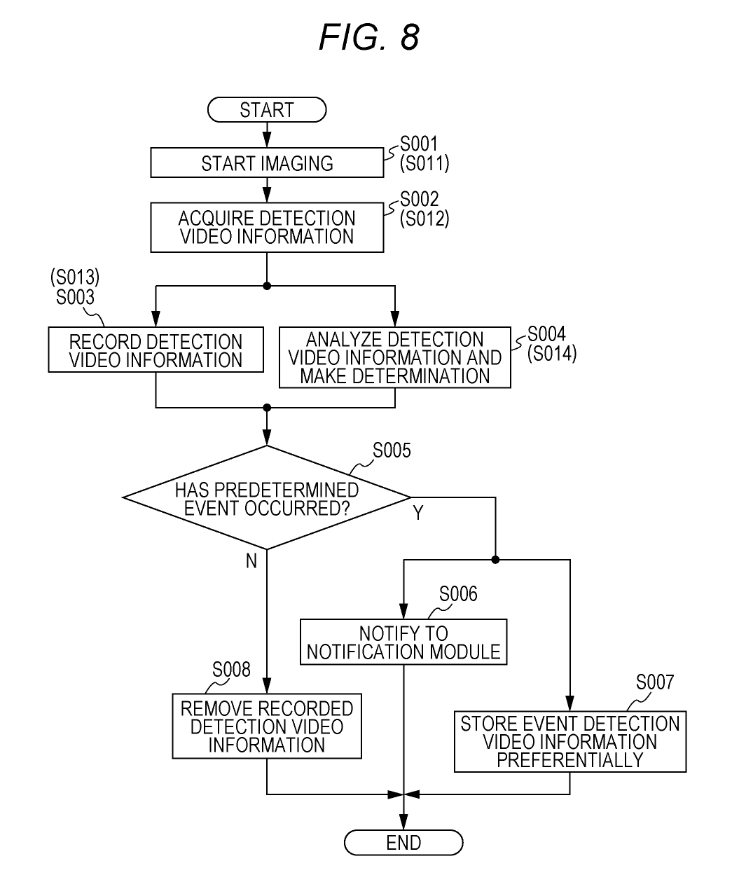

Next, a use form of the nurse call system 10 according to this embodiment will be described. FIG. 8 is a flowchart illustrating an example of a use form of the nurse call system 10. In description of the use form, the configuration illustrated in FIG. 2 is appropriately used.

As illustrated in FIG. 8, the imaging unit 50 starts imaging a predetermined range (step S001). The imaging starts when a patient is hospitalized and comes to a sickbed first. However, the imaging unit 50 may perform imaging regardless of whether patients are correlated with individual sickbeds. This imaging is performed continuously so that the detection video information is generated continuously, and the detection video information is output to the control unit 40 whenever it is generated.

Subsequently, the control unit 40 acquires the detection video information generated by the imaging unit 50 (step S002).

The determination module 42 temporarily stores all of the acquired detection video information in the storage module 46 (step S003). The determination module 42 also outputs the acquired detection video information to the analysis module 41. The analysis module 41 analyzes the acquired detection video information and outputs the analysis result to the determination module 42. This analysis is performed using the above-described algorithm or the like, whereby the movement information of the patient (subject) is generated. The determination module 42 performs the above-described matching process or the like on the analysis result received from the analysis module 41 to make determination on the occurrence of the predetermined event (step S004). In this way, it is determined whether the movement information of the patient included in the detection video information matches the event movement information. In this determination, an event threshold is set as necessary according to the importance of the event.

When the determination module 42 determines that the predetermined event has occurred (step S005: YES), the control module 43 outputs a notification to the notification module 22 (step S006). In this case, the “notification” means outputting a notification module activation signal to the notification module 22. In parallel with this process, event detection video information which is the detection video information before and after the occurrence of the predetermined event is preferentially stored and this process ends (step S005: YES, step S007). Here, “preferential storage” means storing the event detection video information in the storage unit 60 with a storage priority value applied thereto.

When the determination module 42 determines that the predetermined event has not occurred (step S005: NO), the detection video information stored in the storage module 46 is removed (step S008) and the process ends.

The process illustrated in the drawing ends in relation to a video of a predetermined period generated by the imaging unit 50. That is, after a series of processes ends in relation to the video of the predetermined period, when detection video information is further generated by the imaging unit 50, the processes of steps S002 to S008 resume.

[Effects of Nurse Call System 10]

According to the nurse call system. 10 of this embodiment, when it is determined that a predetermined event has occurred, event detection video information is stored and protected in the storage unit 60. In this way, the event detection video information that is to be used as a history can be automatically stored in the storage unit 60 based on the determination on the occurrence of the predetermined event. As a result, a tumbling accident of a patient in a hospital causes a dispute later, it is possible to understand the situation before and after the occurrence of the tumbling accident from the stored event detection video information, which can be helpful to correct judgment. Moreover, the movement information of the patient included in the event detection video information can be used as a guideline for treatment after the occurrence of the event. Further, even when the storage unit 60 has a function of automatically removing the information stored in the storage unit 60, since the event detection video information is protected based on the storage priority value, it is possible to prevent important event detection video information from being removed automatically. In this way, the event detection video information which is to be used later after being stored in the storage unit 60 can be stored for a long period.

### Second Embodiment

### Nurse Call System 10

A configuration of a nurse call system according to this embodiment will be described with reference to the drawings. This description will be given in the following order: Functional configuration of nurse call system; Overall configuration of nurse call system; and Hardware configuration for realizing functional configuration.

[Functional Configuration of Nurse Call System. 10]

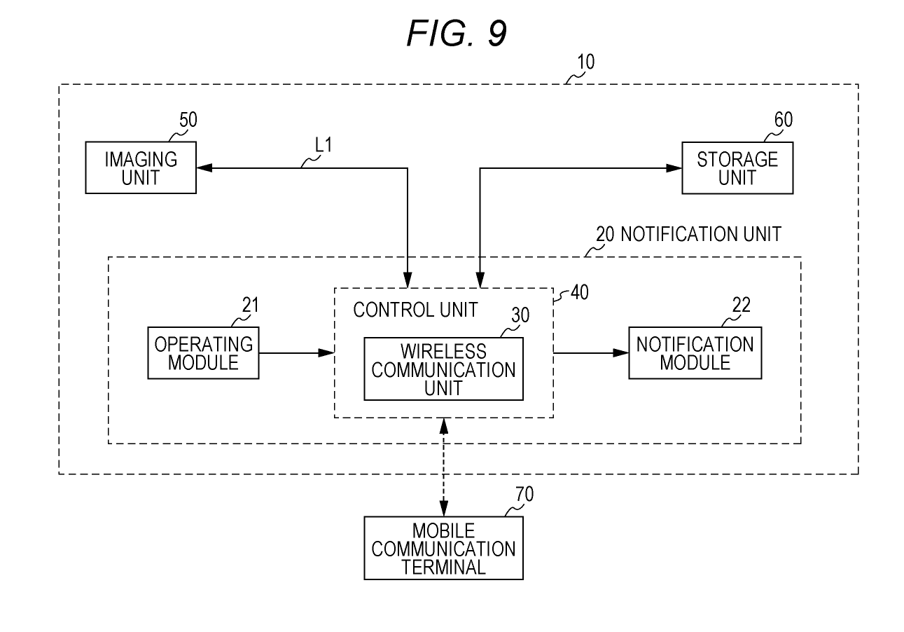

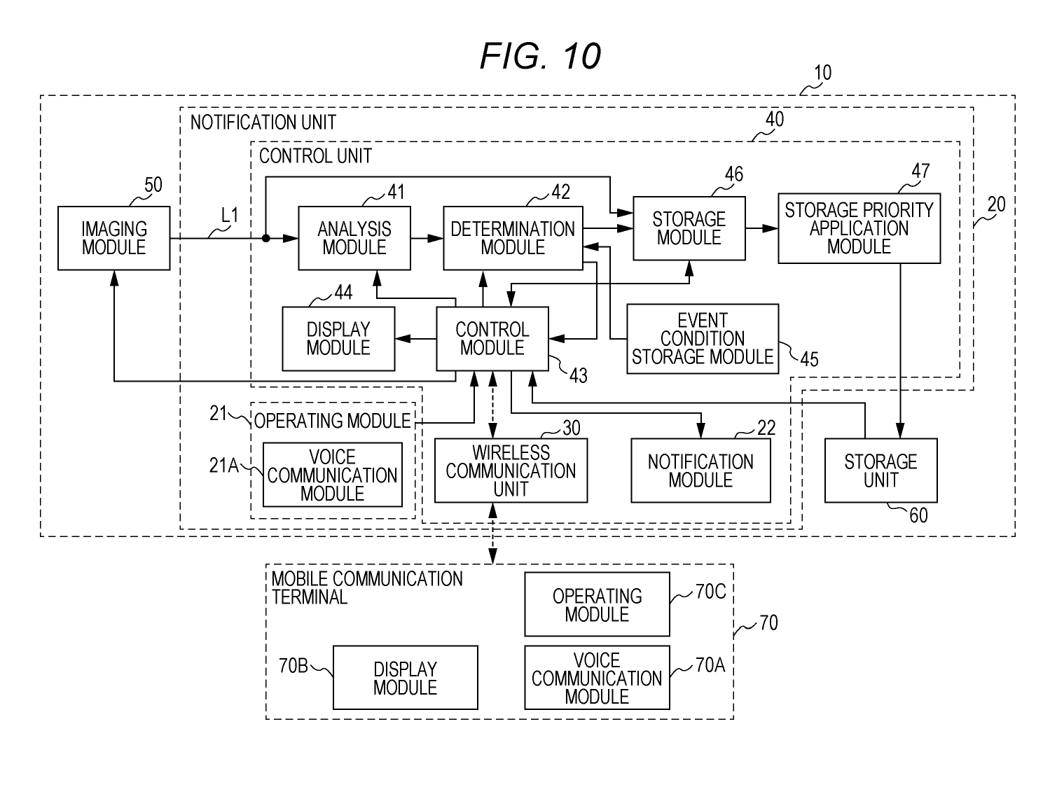

FIGS. 9 and 10 are functional block diagrams illustrating an example of a nurse call system according to this embodiment. FIG. 10 illustrates an example of a detailed configuration of a nurse call system 10 illustrated in FIG. 9. In the drawings, broken-line arrows indicate wireless connections and the same is true herein below.

As illustrated in FIG. 9, the nurse call system 10 is configured to be able to output and input (transmit and receive) information to and from a mobile communication terminal 70 due to a wireless communication unit 30 included in the control unit 40. Besides this, the nurse call system 10 has the same configuration as the nurse call system of the first embodiment.

<Wireless Communication Unit 30>

As illustrated in FIG. 10, the wireless communication unit 30 is provided so as to be able to transmit and receive information to and from the control module 43. The wireless communication unit 30 communicates with the mobile communication terminal 70 by being controlled by the control module 43. The wireless communication unit 30 may be an arbitrary element as long as it has a wireless communication function. The communication form illustrated as an example of the wireless communication can be appropriately used for the wireless communication unit 30.

<Control Module 43>

When a notification module activation signal is output to the notification module 22, the control module 43 transmits (outputs) information indicating the output of the notification module activation signal to the mobile communication terminal 70 via the wireless communication unit 30. The information indicating the output of the notification module activation signal may include information indicating the type of the output notification module activation signal. Examples of the type of the notification module activation signal include a signal based on an operation of the operating module 21 and a signal based on the determination of the occurrence of the predetermined event. Moreover, the information indicating the output of the notification module activation signal may include patient information specified from the notification module activation signal based on the correlation information illustrated in FIG. 6.

<Operating Module 21>

A voice communication module 21A is provided in the operating module 21. The voice communication module 21A is configured to output and input audio information to and from the mobile communication terminal 70 via the control module 43 and the wireless communication unit 30. The voice communication module 21A includes at least a unit for collecting sound and a unit for outputting sound, for example. A specific example thereof is a combination of a microphone and a speaker.

(Mobile Communication Terminal 70)

The mobile communication terminal 70 is configured to be able to transmit and receive information by being connected to the nurse call system 10 via wireless communication. The mobile communication terminal 70 includes a voice communication module 70A, a display module 70B, and an operating module 70C. The mobile communication terminal 70 can receive event occurrence information, event detection video information, detection video information, and the like, for example, from the control unit 40 and can display characters and videos on the display module 70B based on these items of information.

Those described as examples of the mobile communication terminal can be appropriately selected as the mobile communication terminal 70, and a mobile phone, a smartphone, or the like capable of displaying videos and making telephone calls is preferred.

<<Voice Communication Module 70A>>

The voice communication module 70A is configured to be able to output and input audio information to and from the voice communication module 21A. The voice communication module 70A is configured similarly to the voice communication module 21A, for example.

<<Display Module 70B>>

The display module 70B is configured to be able to display various items of information output from the control unit 40. Moreover, a live video of a patient being imaged by the imaging unit 50 may be displayed on the display module 70B. Moreover, the voice communication module 70A and the display module 70B are configured to be used simultaneously. Due to this, a healthcare provider (for example, a nurse) who is a terminal operator can make a telephone call while viewing the live video of a patient displayed on the display module 70B when making a telephone call with the patient using the voice communication module 70A. As a result, the healthcare provider can check the patient's state via visual and auditory senses at a place remote from the patient. The display module 70B corresponds to a “second display module” of the present invention.

<<Operating Module 70C>>

The operating module 70C causes a control module (not illustrated) included in the mobile communication terminal 70 to perform control based on an input operation. The operation is input when a healthcare provider selects a specific process from a list of processes displayed on the display module 70B and the control is executed by the selecting.

As an example, when event occurrence information is transmitted from the control unit 40 to the mobile communication terminal 70, information for prompting selection of a process is displayed on the display module 70B of the mobile communication terminal 70. The selected process may be a process for making a telephone call with a patient to which it is determined that the predetermined event has occurred while viewing the live video and a process for checking the event detection video, for example.

[Overall Configuration of Nurse Call System 10]

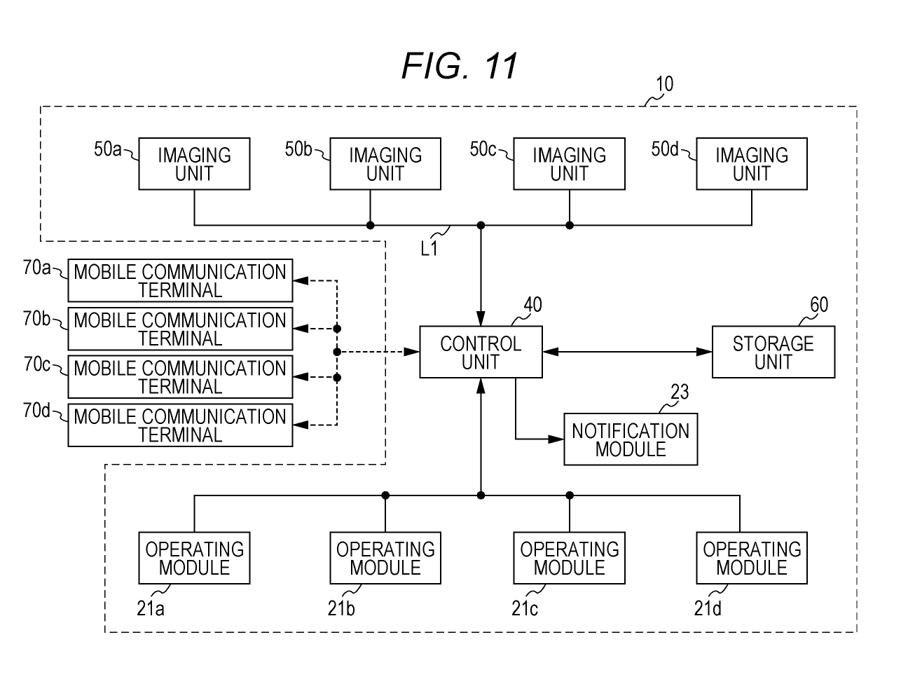

Next, an overall configuration of the nurse call system 10 according to this embodiment will be described. FIG. 11 is a block diagram illustrating an example of an overall configuration of the nurse call system 10 according to this embodiment.

As illustrated in FIG. 11, a plurality of mobile communication terminals 70a to 70d are connected to the control unit 40 so as to be able to transmit and receive information. The other constituent elements are the same as those of the first embodiment. The mobile communication terminals 70a to 70d, the imaging unit 50a to 50d, and the operating modules 21a to 21d are correlated with each other in a manner similarly to the above. As a result, the configuration corresponding to the imaging unit 50a is the same as the configuration illustrated in FIG. 9, for example.

Correlation information among the sickbed number, the imaging unit 50, the operating module 21, and the mobile communication terminal 70 is stored in advance in the storage unit 60 or the like, for example. The correlation information is represented by the correlation of the identifiers (UIDs) applied thereto.

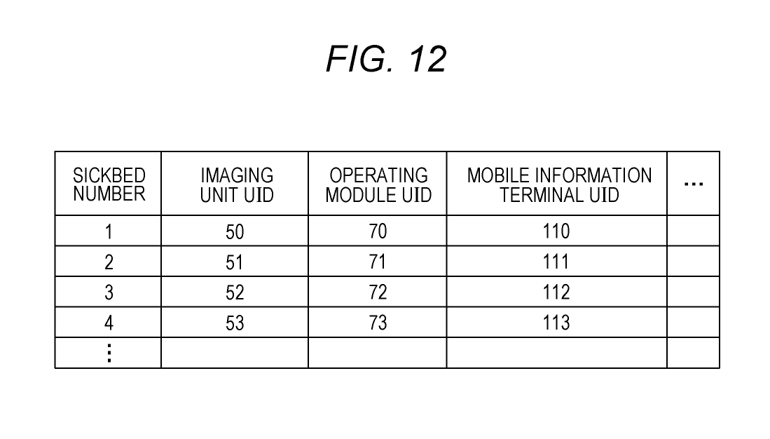

FIG. 12 is a table illustrating an example of the imaging unit 50, the operating module 21, and the mobile communication terminal 70 corresponding to the sickbed number. As illustrated in FIG. 12, the correlation table illustrates the correlation among the sickbed number, the imaging unit UID, the operating module UID, and a mobile communication terminal UID (hereinafter sometimes referred to simply as a “mobile communication terminal UID”) applied to each mobile communication terminal 70.

The control unit 40 specifies the mobile communication terminal 70 that transmits “event occurrence information” based on the correlation and transmits “event occurrence information,” “event detection video information,” and the like to the specified mobile communication terminal 70. The specified mobile communication terminal 70 displays a video based on the “event detection video information” on the display module 70B, for example, in response to these items of information.

An example in which the control unit 40 outputs the event occurrence information to the specific mobile communication terminal 70 will be described using the table illustrated in FIG. 12.

First, as a first example, a case in which the detection video information is generated by the imaging unit 50a will be described. In this case, the imaging unit UID “50” is associated with the detection video information. Upon receiving the detection video information, the control unit 40 specifies the sickbed number “1” the mobile communication terminal UID “110” corresponding to the detection video information based on the associated imaging unit UID “50” and the correlation information. When it is determined that the predetermined event has occurred, the control unit 40 outputs event detection video information or the like to the mobile communication terminal 70a corresponding to the mobile communication terminal UID “110” in association with the information of sickbed number “1.”

As a second example, a case in which a notification module activation signal is output from the operating module 21a will be described. In this case, the operating module UID “70” is associated with the signal. Upon receiving the signal, the control unit 40 specifies the sickbed number “1,” the imaging unit UID “50,” and the mobile communication terminal UID “110” corresponding to the signal based on the operating module UID “70” and the correlation information. Upon receiving the signal, the control unit 40 generates an event detection video from the detection video information generated by the imaging unit 50a corresponding to the imaging unit UID “50.” Further, the control unit 40 outputs the event detection video information to the mobile communication terminal 70a in association with the information of sickbed number “1.” The mobile communication terminal 70a displays the event detection video on the display module 70B together with the character of sickbed number “1” in response to the information.

The nurse call system 10 of this embodiment can cause the mobile communication terminal 70 to output a notification via the control unit 40. Thus, the notification module 22 may be omitted from the configuration of the nurse call system 10.

[Hardware Configuration of Nurse Call System 10]

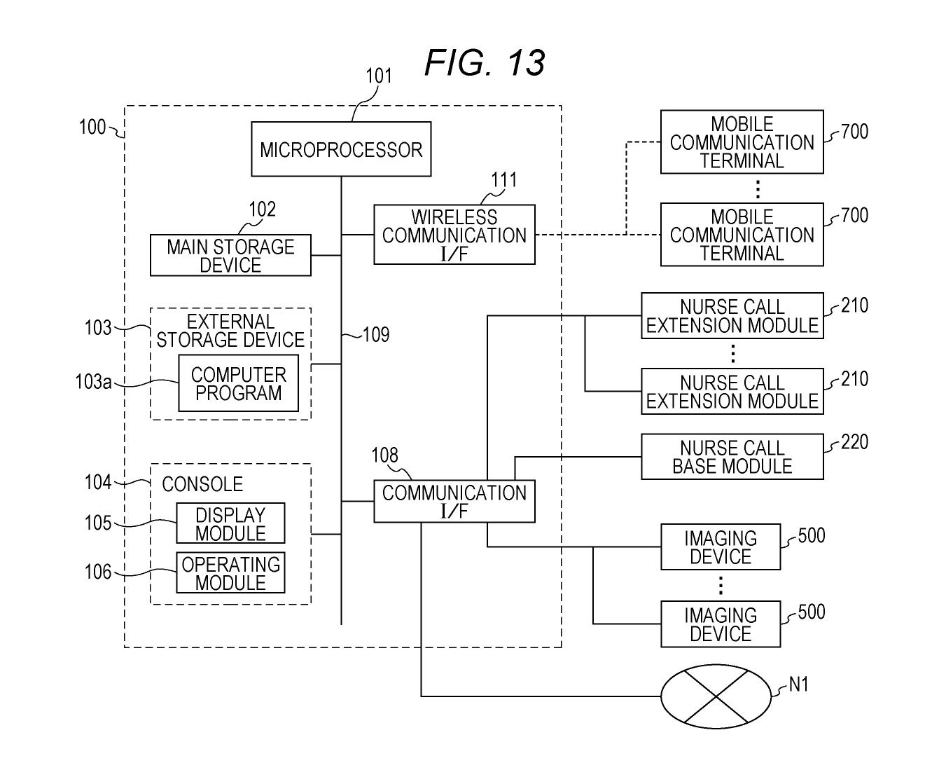

Next, a hardware configuration of the nurse call system 10 (mainly a computer 100) for realizing the above-described functions will be described with reference to FIG. 13. FIG. 13 is a block diagram illustrating an example of a hardware configuration of the nurse call system 10 according to this embodiment.

In the hardware configuration of the nurse call system 10 of this embodiment, a plurality of mobile communication terminals are connected via a wireless communication interface (I/F) 111 so as to be able to communicate with each other. Besides this, the nurse call system 10 has the same configuration as the nurse call system 10 of the first embodiment.

The wireless communication interface 111 is an interface that connects at least the computer 100 and the mobile communication terminal 700. As illustrated in FIG. 13, the wireless communication interface 111 may be provided independently from the communication interface 108, for example. Moreover, the communication interface 108 may be a constituent element that also serves as the wireless communication interface 111. The wireless communication interface 111 realizes the function of the wireless communication unit 30 illustrated in FIG. 9, for example.

The mobile communication terminal 700 includes a voice communication device (not illustrated) having the function of the voice communication module 70A, a display device (not illustrated) having the function of the display module 70B, and an operating device (not illustrated) having the function of the operating module 70C.

The wireless communication form may be an ad-hoc mode in which terminals and devices communicate in parallel, for example, and an infrastructure mode in which the computer 100 is used as a base module and terminals and devices are used as extension modules, for example. Examples of the wireless communication interface (I/F) 111 include a wireless LAN compliant with IEEE 802.11 series and IEEE 802.15 series.

The configuration of the nurse call base module 220 corresponding to the notification module 22 may be omitted from the hardware configuration.

[Use Form of Nurse Call System 10]

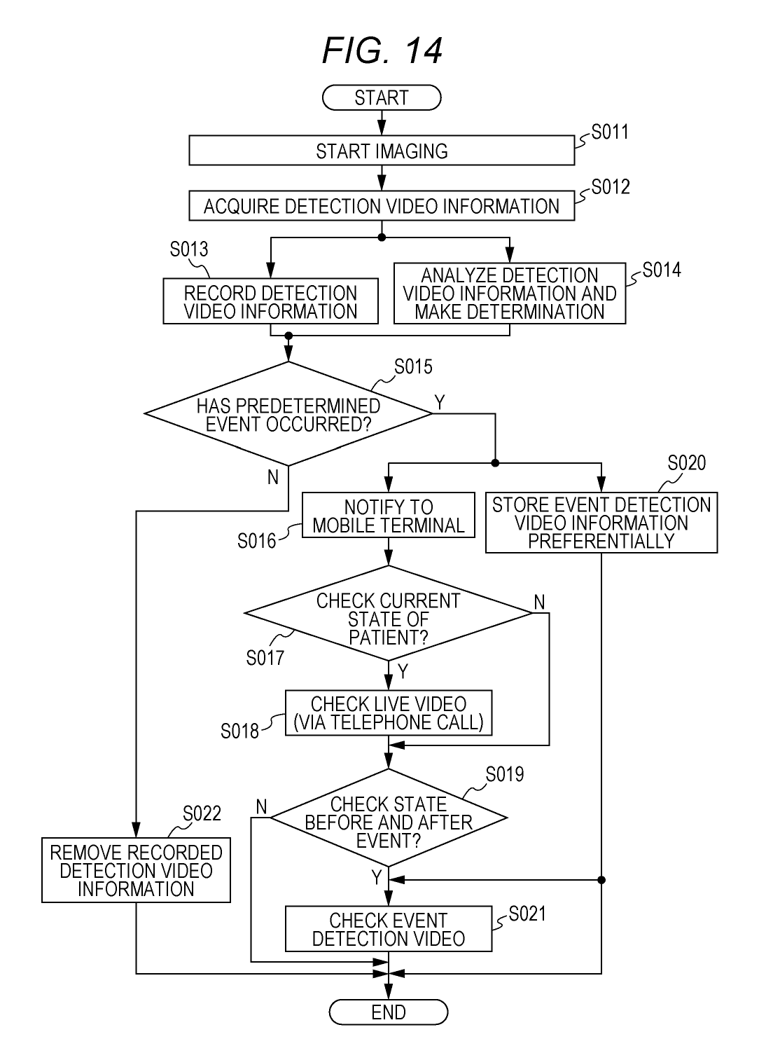

Next, a use form of the nurse call system 10 according to this embodiment will be described. FIG. 14 is a flowchart illustrating an example of a use form of the nurse call system 10. In description of the use form, the flowchart illustrated in FIG. 8 and the functional block illustrated in FIG. 10 are appropriately used.

As illustrated in FIG. 14, this flowchart illustrate the processes that the control unit 40 outputs a notification to the mobile communication terminal 70 only when it is determined that the predetermined event has occurred among the processes illustrated in FIG. 8.

The processes of steps S011 to S015 are performed similarly to the processes of steps S001 to S005 of the flowchart illustrated in FIG. 8 (steps S011 to S015).

When the determination module 42 determines that the predetermined event has occurred (step S015: YES), the control module 43 outputs a notification to the mobile communication terminal 70 (step S016). In this case, the “notification” means outputting event occurrence information to the mobile communication terminal 70 via the wireless communication interface 111. The mobile communication terminal 70 notifies the external device of the occurrence of the predetermined event in response to the notification. This notification may be realized in an arbitrary form as long as it can be sensed by the five senses of a person and may be realized via sound, characters, videos, vibration, and the like, for example. In parallel with this process, the control module 43 preferentially stores the “event detection video information” which is the detection video information of a period before and after the occurrence of the predetermined event in the storage unit 60 or the like (step S015: YES, step S020).

A terminal operator selects a subsequent process in response to the notification. This selection is performed by the terminal operator using the operating module 70C. When “Check the current state of a patient” is selected as the subsequent process (step S017: YES), the control unit 40 displays a video including the patient, generated by the corresponding imaging unit 50 on the display module 70B on a real-time basis. Further, the terminal operator makes a telephone call with the patient using the voice communication module 70A as necessary (step S018) and the flow proceeds to step S019. On the other hand, when the terminal operator does not select to check the current state of the patient (step S017: NO), the flow proceeds to step S019.

In step S019, it is determined whether “Check the state before and after the occurrence of the event” is selected as the subsequent process. In this case, the “state before and after occurrence of the event” is the video information before and after the occurrence of the event.

When the terminal operator selects to check the state before and after occurrence of the event (step S019: YES), the “event detection video information” stored in the storage unit 60 or the like is acquired in step S020, the “event detection video” based on the event detection video information is displayed on the display module 70B, and this process ends (step S021). Moreover, steps S017 to S018 may be replaced with step S019 as necessary.

On the other hand, when the determination module 42 determines that the predetermined event has not occurred (step S015: NO), the detection video information stored in the storage module 46 is removed and the process ends (step S022).

The process may be performed by substituting the mobile communication terminal 70 with the control unit 40. For example, an operating module (not illustrated) may be provided in the control unit 40, the process may be selected in the above-described manner, and the patient's state may be displayed on the display module 44. Moreover, a voice communication module (not illustrated) may be provided in the control unit 40, and the operator may make a telephone call with a patient while displaying a patient's state on the display module 44.

In the nurse call system 10 of this embodiment, the event occurrence information may be output to the notification module 22 and the mobile communication terminal 70. In this case, the process when YES is obtained in step S015 of the process illustrated in FIG. 14 is performed in parallel with the process (step S006) of outputting a notification to the notification module 22 illustrated in FIG. 8, for example.

The other features of the use form of the nurse call system 10 according to this embodiment are the same as those of the use form of the nurse call system 10 according to the first embodiment.

### EXAMPLE

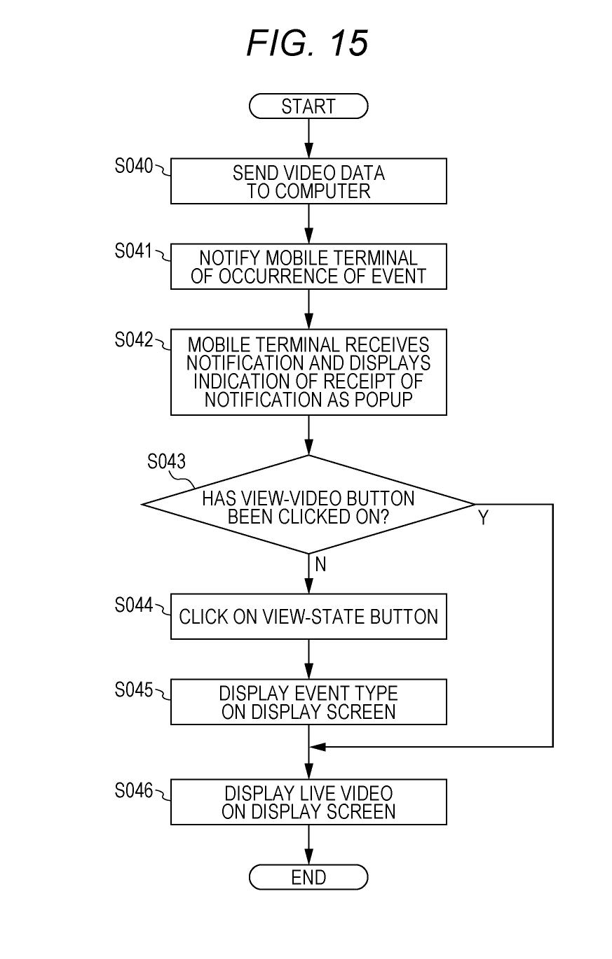

FIG. 15 is a flowchart illustrating a use form of a nurse call system according to Example of the nurse call system 10 of this embodiment. This nurse call system 10 is configured using hardware components corresponding to the respective functions. Examples of the hardware components include the configuration illustrated in FIG. 13. The flow of operations according to Example will be described appropriately with reference to FIG. 13.

First, video data imaged by the imaging device 500 is transmitted to the computer 100 (step S040).

Subsequently, the computer 100 analyzes the acquired video, and when it is determined that a predetermined event has occurred, notifies the mobile communication terminal 700 of the occurrence of the event (step S041). In this case, the “notification” means outputting the event occurrence information to the mobile communication terminal 70 via the wireless communication interface 111.

Subsequently, the mobile communication terminal 700 displays information indicating that the notification (event occurrence information) has been received on a display screen. This information is displayed as a popup icon (step S042).

Subsequently, the terminal operator who operates the mobile communication terminal 700 selects a “view-video button” included in the information indicating the reception of the event occurrence information by clicking or tapping (step S043: YES), and a live video of the patient, imaged by the imaging device 500 is displayed on the display screen and the process ends (step S046).

When the terminal operator selects a “view-state button” included in the information (step S044) rather than selecting the “view-video button” (step S043: NO), a type corresponding to the event is displayed on the display screen (step S045). Further, the live video of the patient, imaged by the imaging device 500 is displayed on the display screen (step S046), and the process ends.

[Effects of Nurse Call System 10]

The nurse call system 10 of this embodiment has the same configuration as the first embodiment except that the nurse call system 10 includes the mobile communication terminal 70 as a notification unit. Thus, the same effects as the first embodiment are obtained. Further, a healthcare provider who uses the nurse call system 10 of this embodiment can be informed of an abrupt change in the condition of the patient through the mobile communication terminal 70 at a place remote from the patient, for example. Moreover, when the mobile communication terminal 70 is configured to be able to display the detection video of the patient and to make a telephone call with the patient, the healthcare provider can understand the patient's state visually and through the telephone call at a remote place.

### Third Embodiment

### Nurse Call System 10

A configuration of a nurse call system according to this embodiment will be described with reference to the drawings. The description will be given in the following order: Functional configuration of nurse call system; Overall configuration of nurse call system; and Hardware configuration for realizing functional configuration.

[Functional Configuration of Nurse Call System 10]

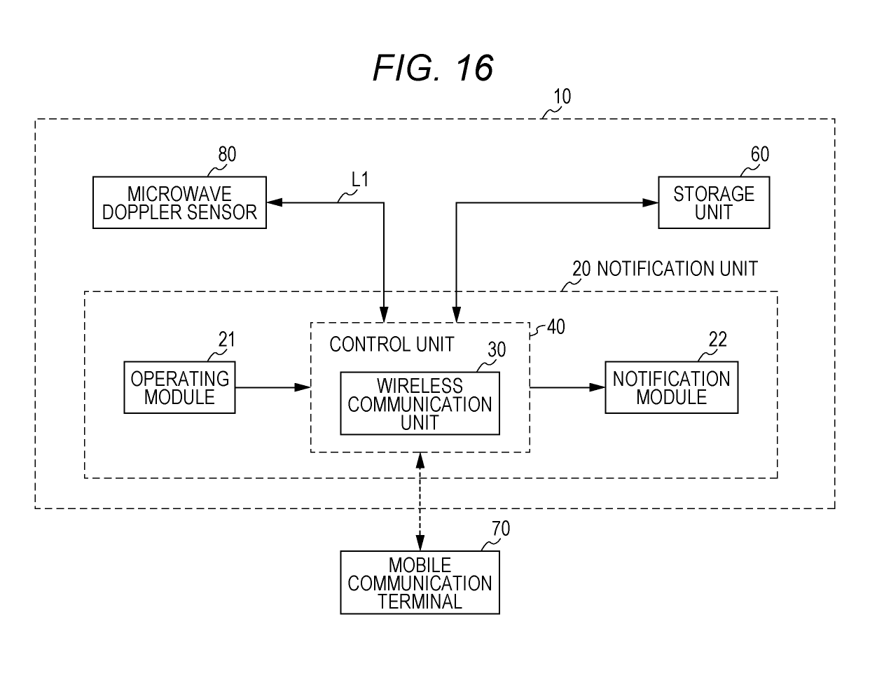

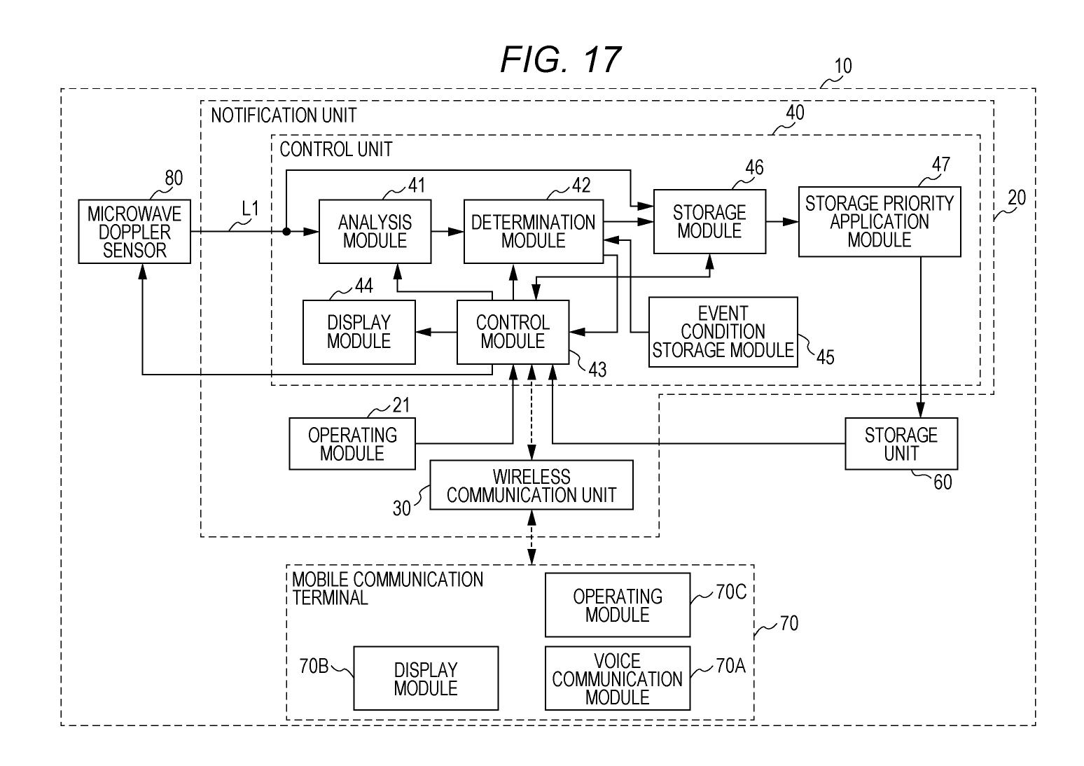

FIGS. 16 and 17 are functional block diagrams illustrating an example of a nurse call system according to this embodiment. FIG. 17 illustrates an example of a detailed configuration of a nurse call system 10 illustrated in FIG. 16.

As illustrated in FIGS. 16 and 17, the nurse call system 10 includes a microwave Doppler sensor 80 as a detection unit. The other constituent elements have the same configurations as those of the nurse call system of the second embodiment.

(Microwave Doppler Sensor 80)

The microwave Doppler sensor 80 continuously generates body motion data (a respiratory state or a daily rhythm) of a patient who is a subject. The microwave Doppler sensor 80 includes a transmitter antenna (transmitting module), a receiver antenna (receiving module), and a phase detector which are not illustrated.

The transmitter antenna is connected to an oscillator having a certain frequency to transmit radio waves to the subject. The receiver antenna receives radio waves (reflected waves) reflected from the subject to output the reflected waves to the phase detector. The phase detector combines a signal indicating the transmitted radio waves and a signal indicating the received radio waves. As signal output from the phase detector as the result of the signal combination has a highest intensity when the transmitted radio waves have the same phase as the received radio waves and has a lowest intensity when the two types of radio waves have the opposite phases. Thus, when the subject is moving, an interference fringe appears in the signal output from the phase detector (this is the Doppler shift effect). In this way, the microwave Doppler sensor 80 can detect a body motion by using the Doppler shift effect. Examples of the body motion of the patient, detected by the microwave Doppler sensor 80 include a respiratory state, a heart beat state, and the like.

The microwave Doppler sensor 80 detects the body motion by receiving radio waves reflected from the body surface (a portion including the chest, for example) of a patient. The radio waves used for detecting the body motion can pass through clothes, a blanket, and the like. Thus, the body motion can be detected without a large influence of the movement of clothes, a blanket, or the like, and falling of an object such as a blanket or clothes other than the patient is rarely detected as a body motion. Thus, it is possible to selectively detect the body motion of the patient.

<<Control Unit 40>>

Although the control unit 40 has the same overall configuration as that of the first to second embodiment, the analysis module 41 that specifies the detection information may be omitted as necessary. This is because the body motion information is generated by the microwave Doppler sensor 80. Moreover, the control unit 40 includes the determination module 42. The determination module 42 detects a predetermined event from the body motion information generated by the microwave Doppler sensor 80.

<Determination Module 42>

The determination module 42 detects the predetermined event based on known frequency analysis such as FFT analysis or wavelet analysis. This analysis is performed in such a way that a body motion frequency related to the respiration is specified from features of the signal frequency and the respiratory state is estimated from the body motion frequency, for example. The determination module 42 determines whether the body motion frequency is within a certain frequency range, for example. When a body motion frequency that is out of the certain frequency range is detected, the determination module 42 determines that a predetermined event has occurred. A specific example of the body motion frequency which results in such a determination includes a body motion frequency in an apnea condition, which is a very low frequency and a body motion frequency in a hyperpnea, which is a very high frequency.

When a very large signal variation is detected in the analysis, the determination module 42 determines that a movement (tossing or the like), a body motion, or the like other than the respiration has occurred to the subject. A large signal variation is detected by setting a threshold to a time-derivative of signals, for example.

Although the determination of the determination module 42 has been described by way of an example of a specific movement, the determination module 42 may determine the occurrence of the predetermined event by matching the body motion information with the body motion information of the patient, received from the microwave Doppler sensor 80.

<Control Module>

When the occurrence of the predetermined event is detected, the control module 43 preferentially stores the “event detection body motion information” which is the body motion information before and after the occurrence of the predetermined event in the storage unit 60 or the like. The “event detection body motion information” is the body motion information including the body motion indicating the occurrence of the predetermined event. The body motion information including the body motion indicating the occurrence of the predetermined event is the body motion information generated by the microwave Doppler sensor 80 in a predetermined period including the event occurrence time. The starting point of the predetermined period is the time occurring a first predetermined period earlier than the event occurrence time and the ending point thereof is the time occurring a second predetermined period after the event occurrence time. Moreover, the body motion information of a predetermined period may be generated in the above-described manner using the time at which the operation of the operating module 21 is input to the control module 43 as the event occurrence time, for example.

The nurse call system 10 may include a plurality of detection unit. In this case, the imaging unit 50 and the microwave Doppler sensor 80 are used as the plurality of detection unit, for example. The imaging unit 50 and the microwave Doppler sensor 80 monitor the corresponding patient and generate the movement information thereof. The movement information of the patient, generated by the plurality of detection unit is analyzed by the control unit 40, whereby the occurrence of the predetermined event is determined. By detecting the movement information using a plurality of detection unit, it is possible to improve the determination accuracy of the occurrence of the predetermined event.

When the imaging unit 50 and the microwave Doppler sensor 80 are used as the plurality of detection unit, the control unit 40 generates the movement information of the patient using any one of the plurality of detection unit. In this case, the occurrence of the predetermined event is determined based on the detection information detected by the microwave Doppler sensor 80, for example. The detection video information generated by the imaging unit 50 is used for displaying the event detection video when it is determined that the predetermined event has occurred. In this case, since the control unit 40 does not perform image analysis, the load of a computer or the like used as the control unit 40 can be reduced.

An event occurrence time may be used as a trigger for the analysis of the detection video information of the control unit 40 based on detection information detected by another detection unit. When it is determined that the occurrence of the predetermined event has occurred, a moving image including the occurrence time thereof is acquired from the storage unit 60, and the moving image is analyzed by the control unit 40, whereby the occurrence of the predetermined event is verified. By doing so, since the control unit 40 does not regularly perform the image analysis, the load of the computer or the like used as the control unit 40 can be reduced.

The nurse call system 10 of this embodiment can output a notification to the mobile communication terminal 70 via the control unit 40. Thus, the notification module 22 may be omitted from the configuration of the nurse call system 10.

[Overall Configuration of Nurse Call System 10]

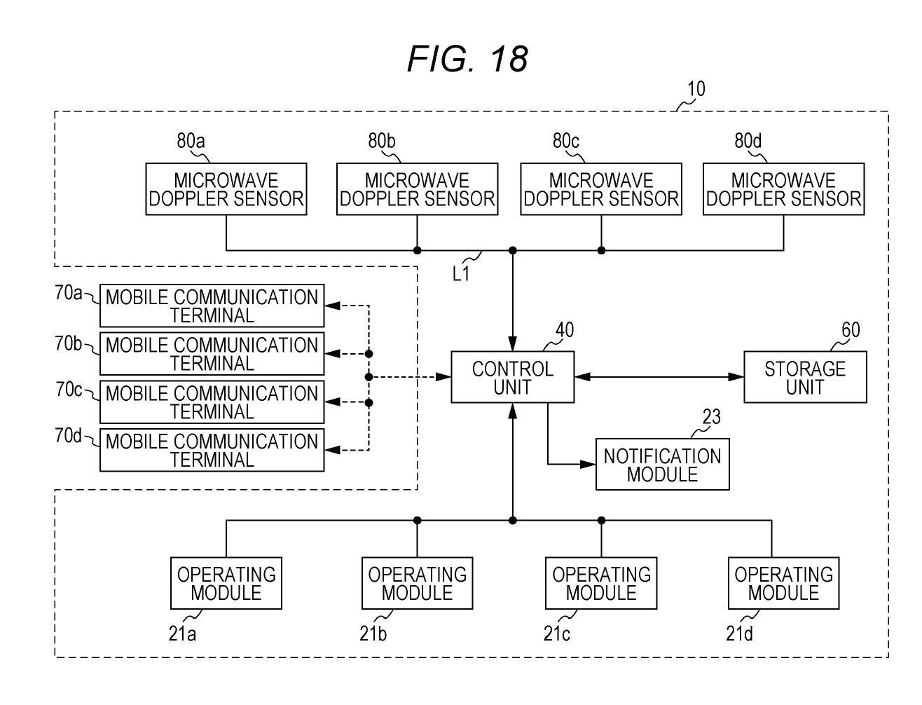

Next, an overall configuration of the nurse call system 10 according to this embodiment will be described. FIG. 18 is a block diagram illustrating an example of an overall configuration of the nurse call system 10 according to this embodiment.

As illustrated in FIG. 18, a plurality of microwave Doppler sensors 80 as the detection unit is connected to the control unit 40. A microwave Doppler sensor 80a is correlated with the operating module 21a and the mobile communication terminal 70a and is connected to the control unit 40 so as to be able to output and input information. In this way, the microwave Doppler sensor 80a is configured similarly to the nurse call system 10 illustrated in FIG. 16. The same is true for the microwave Doppler sensors 80b to 80d.

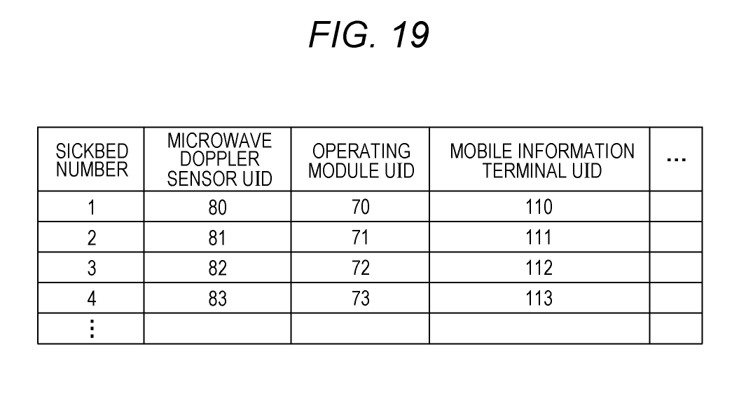

FIG. 19 is a table illustrating an example of the microwave Doppler sensor 80, the operating module 21, and the mobile communication terminal 70 corresponding to the sickbed number.

As illustrated in FIG. 19, this correlation table illustrates a correlation among the sickbed number, a UID (hereinafter sometimes referred to simply as a “microwave Doppler sensor UID”) applied to each microwave Doppler sensor 80, the operating module UID, and the mobile communication terminal UID. The information correlated with the sickbed number is not limited to these items of information, but those illustrated in the tables of FIGS. 6 and 12 may be appropriately correlated with the sickbed number, for example.

When the predetermined event is determined from the predetermined body motion information, the control unit 40 can transmit the event occurrence information and the like to the corresponding mobile communication terminal 70 from the correlation between the microwave Doppler sensor 80 and the mobile communication terminal 70. Further, the control unit 40 can output a notification to the external device by controlling the mobile communication terminal 70.

[Hardware Configuration of Nurse Call System 10]

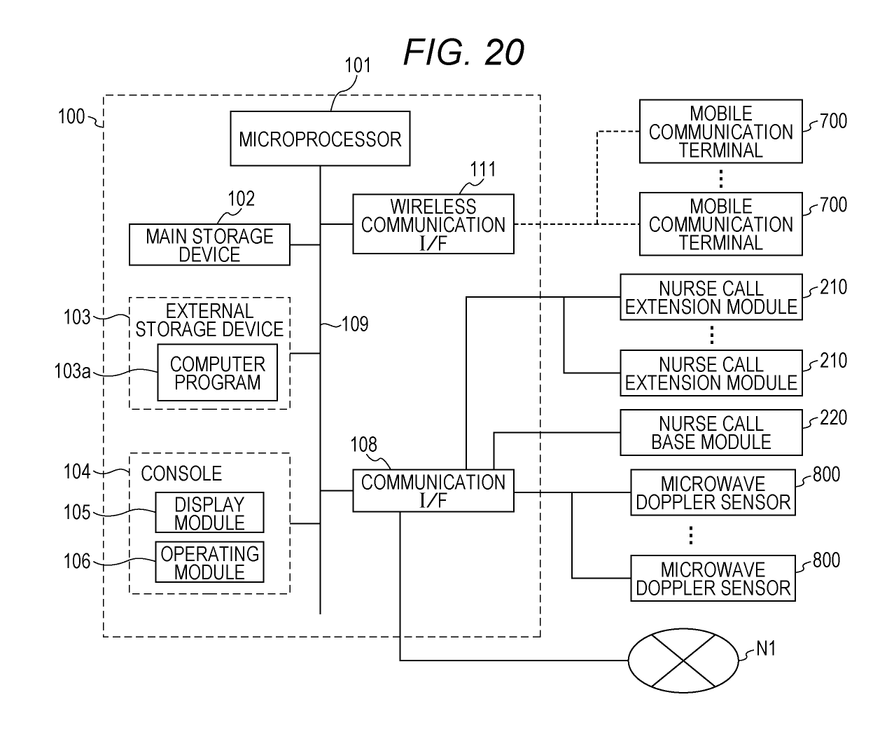

Next, a hardware configuration of the nurse call system 10 (mainly a computer 100) for realizing the above-described functions will be described with reference to FIG. 20. FIG. 20 is a block diagram illustrating an example of a hardware configuration of the nurse call system 10 according to this embodiment.

The hardware configuration of the nurse call system 10 of this embodiment includes a plurality of microwave Doppler sensors 800 instead of the plurality of imaging devices 500 as the detection unit. The other constituent elements have the same configuration as those of the nurse call system 10 of the second embodiment but may have the same configuration as those of the nurse call system 10 of the first embodiment.

The configuration of the nurse call base module 220 corresponding to the notification module 22 may be omitted from the hardware configuration.

[Use Form of Nurse Call System 10]

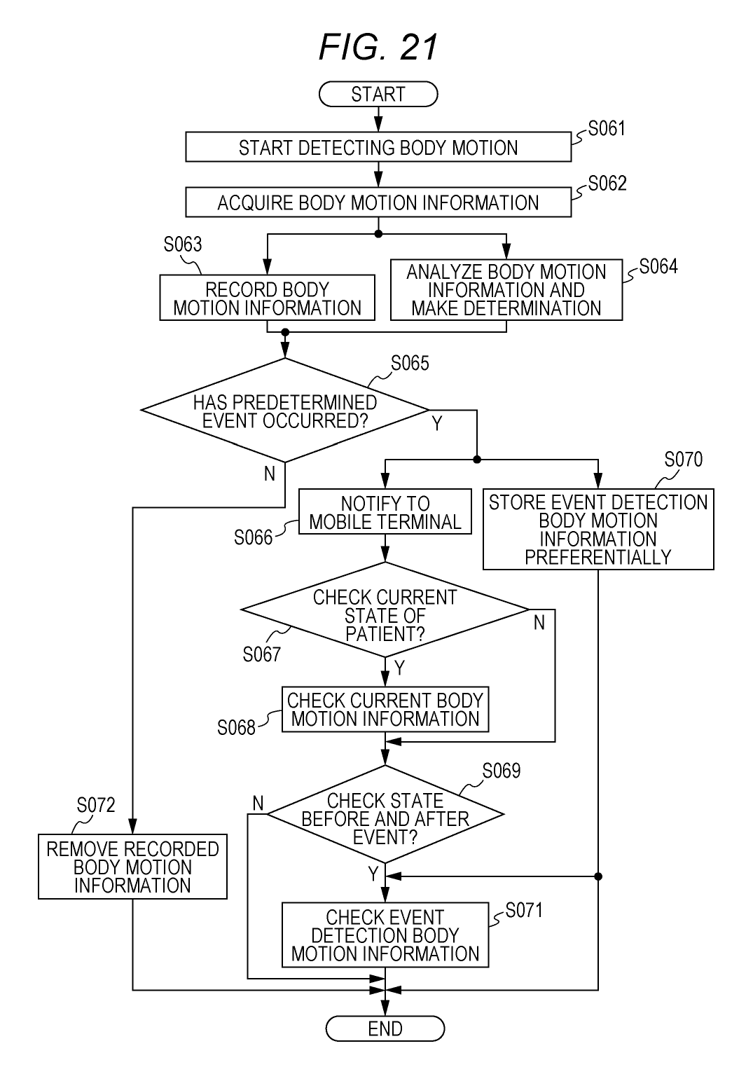

Next, a use form of the nurse call system 10 according to this embodiment will be described. FIG. 21 is a flowchart illustrating an example of a use form of the nurse call system 10. In description of the use form, the flowchart illustrated in FIG. 16 and the functional block illustrated in FIG. 16 are appropriately used.

As illustrated in FIG. 21, in this flowchart, the microwave Doppler sensor 80 is used the detection unit in the process illustrated in FIG. 14. Specifically, the control unit 40 makes a determination on the occurrence of the predetermined event based on the body motion information generated by the microwave Doppler sensor 80. Hereinafter, the respective steps will be described in detail.

The microwave Doppler sensor 80 emits microwaves to a patient to start detecting the body motion. With the body motion detection, the body motion information of the patient is continuously generated and the body motion information is output to the control unit 40 whenever it is generated (step S061).

Subsequently, the control unit 40 acquires the body motion information of the patient output from the microwave Doppler sensor 80 (step S062).

All the acquired body motion information is temporarily stored in the storage module 46 (step S063). In parallel with this, the analysis module 41 analyzes the acquired body motion information as necessary and the determination module 42 determines whether the predetermined event has occurred. With this process, the body motion information of a subject is acquired. The subject is a patient corresponding to the sickbed (step S064). When an event threshold corresponding to the importance of the event is set in the above-described manner, the determination of the predetermined event is executed according to the event threshold.

When the determination module 42 determines that the predetermined event has occurred (step S065: YES), the control module 43 outputs a notification to the mobile communication terminal 70 (step S066). In this case, the “notification” means outputting the event occurrence information to the mobile communication terminal 70 via the wireless communication interface 111. The mobile communication terminal 70 informs the external device of the occurrence of the predetermined event in response to the notification. This notification may be realized in an arbitrary form as long as it can be sensed by the five senses of a person and may be realized via sound, characters, videos, vibration, and the like, for example. In parallel with this process, the control module 43 preferentially stores the “event detection body motion information” which is the body motion information of a period before and after the occurrence of the predetermined event in the storage unit 60 or the like (step S065: YES, step S070).

A terminal operator selects a subsequent process in response to the notification. This selection is performed by the terminal operator using the operating module 70C. When “Check the current state of a patient” is selected as the subsequent process (step S067: YES), the terminal operator displays the body motion information generated by the corresponding imaging unit 50 on the display module 70B on a real-time basis (step S068), and the flow proceeds to step S069. On the other hand, when the terminal operator does not select to “check the current state of the patient” (step S067: NO), the flow proceeds to step S069.

In step S069, it is determined whether “Check the state before and after the occurrence of the event” is selected as the subsequent process. In this case, the “state before and after occurrence of the event” is the body motion information before and after the occurrence of the event.

When “Check the state before and after occurrence of the event” is selected (step S069: YES), the “event detection body motion information” stored in the storage unit 60 or the like is acquired in step S070, the “event detection body motion information” is displayed on the display module 70B, and this process ends (step S071). Moreover, steps S067 to S068 may be replaced with step S069 as necessary.

When the determination module 42 determines that the predetermined event has not occurred (step S065: NO), the body motion information stored in the storage module 46 is removed and the process ends (step S072).

[Effects of Nurse Call System 10]

According to the nurse call system. 10 of this embodiment, the microwave Doppler sensor 80 is provided instead of the imaging unit 50 as the detection unit in the nurse call system of the first or second embodiment. Thus, the same effects as the first and second embodiments are obtained, and a minute body motion such as a respiratory state which is difficult to detect using the imaging unit 50 can be detected.

### Fourth Embodiment

### Nurse Call System 10

A configuration of a nurse call system according to this embodiment will be described with reference to the drawings. The description will be given in the following order: Functional configuration of nurse call system; Overall configuration of nurse call system; and Hardware configuration for realizing functional configuration.

[Functional Configuration of Nurse Call System 10]

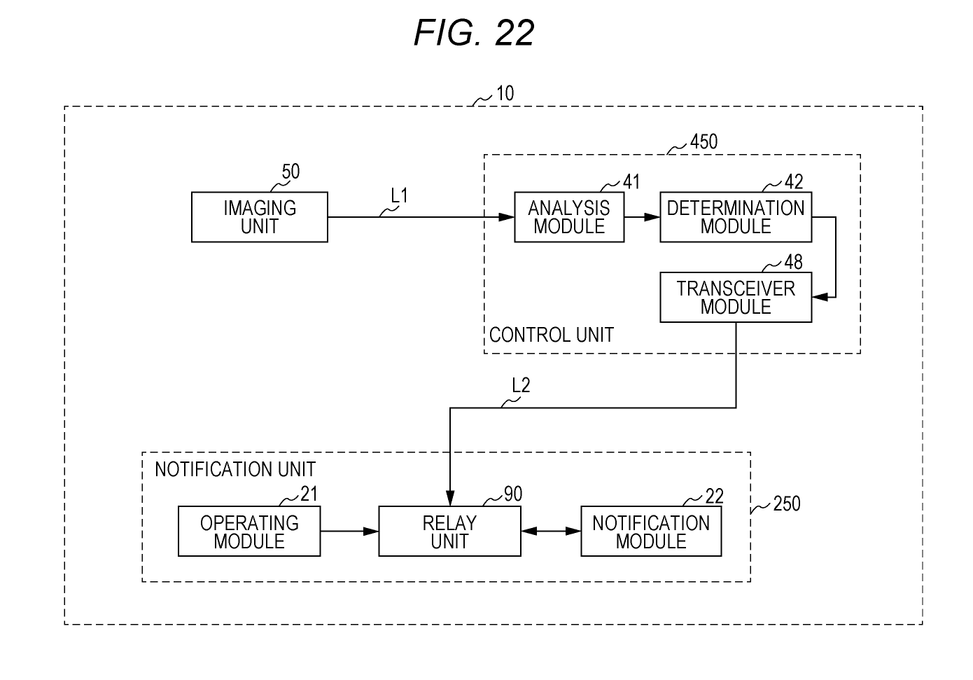

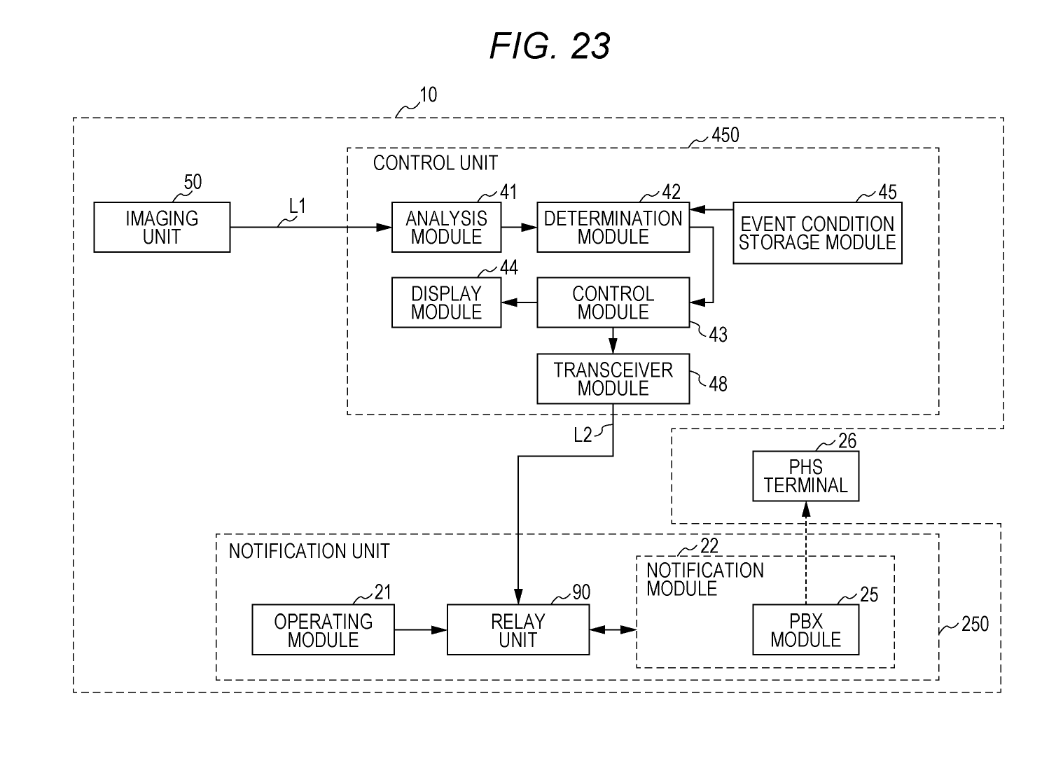

FIGS. 22 and 23 are functional block diagrams illustrating an example of a nurse call system according to this embodiment. FIG. 23 illustrates an example of a detailed configuration of a nurse call system 10 illustrated in FIG. 22.

As illustrated in FIG. 22, the nurse call system 10 of this embodiment includes a notification unit 250 in which the control unit 40 is replaced with a relay unit 90 in the notification unit 20 of the nurse call system 10 of the first embodiment. This “replacement” may involve physical replacement and may involve functional replacement by adding the function of the relay unit 90 and disabling the control unit 40. The control unit 450 is connected to the relay unit 90 provided independently from the notification unit 250 so as to be able to output and input information. Although the control unit 450 may be configured similarly to the control unit 40, the control unit 450 may not include the storage module 46 and the storage priority application module 47 of the control unit 40. The nurse call system 10 of this embodiment may not include the storage unit 60.

(Notification unit 250)

The notification unit 250 is a nurse call device that outputs a notification to a headquarter such as a nurse's station when a patient who is an operator inputs a predetermined operation. The notification unit 250 includes the operating module 21, the notification module 22, and the relay unit 90. The operating module 21 and the notification module 22 are provided so that a signal can be output from at least the operating module 21 to the notification module 22 via the relay unit 90.

For example, when the notification module activation signal is transmitted (output) from the operating module 21 to the notification module 22, the signal is received (input) in the notification module 22 via the relay unit 90 provided therebetween. The notification module 22 operates in response to the input of the signal and a notification is output to the external device based on the signal. The notification module activation signal is transmitted based on the operation of the patient, for example. In this case, the notification module activation signal corresponds to a “first signal.” Moreover, the operating module 21 and the notification module 22 may have the same configuration as those of the first embodiment.

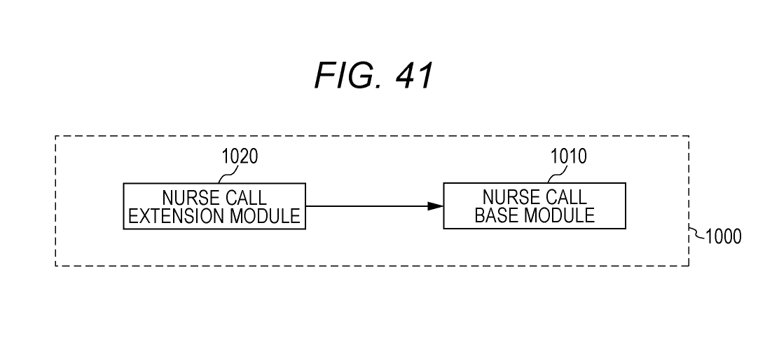

The notification unit 250 can be configured by providing the relay unit 90 to the notification unit such as a conventional existing nurse call device. FIG. 41 is a functional block diagram illustrating a conventional nurse call device 1000. The nurse call device 1000 is configured by connecting a nurse call base module 1010 and a nurse call extension module 1020. Here, the nurse call base module 1010 corresponds to the notification module 22 and the nurse call extension module 1020 corresponds to the operating module 21.

The relay unit 90 illustrated in FIG. 22 is provided between the nurse call extension module 1020 and the nurse call base module 1010 of the conventional nurse call device 1000 whereby the notification unit 250 is formed. Further, by connecting the control unit 450 to the relay unit 90, the nurse call system 10 of this embodiment can be formed. The configuration of the control unit 450 can be appropriately selected from the configurations of the control unit 40 described in the first to third embodiments.

In the first to third embodiments, the control unit 40 may be replaced with the relay unit 90 and the control unit 450 connected to the relay unit 90 may be further included. In this way, the nurse call system 10 of this embodiment can be formed. In this case, the imaging unit 50 is connected to the control unit 450. The transmission and reception of signals among the operating module 21, the notification module 22, and the relay unit 90 may be realized via wireless communication or cable communication.

<<Relay Unit 90>>

The relay unit 90 outputs the signal received from the operating module 21 to the notification module 22. Moreover, the control unit 450 is connected to the relay unit 90 so as to be able to transmit and receive signals (information). The relay unit 90 is connected to the control unit 450 so as to be able to output and input information. The input and output is realized by transmission and reception via communication, for example. The imaging unit 50 is connected to the control unit 450. The imaging unit 50 continuously generates information including the movement information of the patient.

The relay unit 90 may have the function of a path switching unit, may include the function of a signal conversion unit, or may include the functions of both.

As illustrated in FIG. 23, the control unit 450 includes a display module 44 that displays at least a detection video based on the detection video information received from the imaging unit 50, a control module 43 that controls at least the display module 44 based on an input signal, and an event condition storage module 45 that is connected to the determination module 42.

(Control Unit 450)

When the control unit 450 determines that the predetermined event has occurred based on the process of the analysis module 41 and the determination module 42, a transceiver module 48 having received the determination result transmits a notification module activation signal to the relay unit 90. The notification module 22 having received the signal is operated to output a notification to the external device. As a result, a healthcare provider present in a nurse's station, for example, can understand that a certain abnormality has occurred to the patient.

The control unit 450 includes at least the analysis module 41, the determination module 42, and the transceiver module 48, and further includes the control module 43 and the display module 44. The analysis module 41, the determination module 42, the control module 43, and the display module 44 can have the same configurations as those described in the control unit 40.

<<Transceiver Module 48>>

The transceiver module 48 is connected to a transceiver module (not illustrated) provided in the relay unit 90 so as to be able to transmit and receive information. The transceiver module 48 transmits a predetermined signal (information) to the transceiver module (not illustrated) provided in the relay unit 90 by being controlled by the control module 43 based on the determination result received from the determination module 42. The relay unit 90 having received the predetermined signal outputs a notification module activation signal to the notification module 22. The predetermined signal corresponds to a “second signal” of the present invention. The notification module 22 receives the notification module activation signal and operates based on the notification module activation signal whereby a notification is output to the external device. In this case, the notification module activation signal corresponds to a “third signal” of the present invention.

The transceiver module 48 may be an arbitrary element as long as it has at least the configuration as a transmitting module that can transmit signal to the relay unit 90. The transmission form of the transceiver module 48 may be wireless communication or cable communication. The signal transmitted to the relay unit 90 corresponds to the second signal of the present invention. The signal transmitted to the relay unit 90 may be appropriately selected from those examples described as the second signal. The transceiver module 48 corresponds to a “transceiving unit” of the present invention.

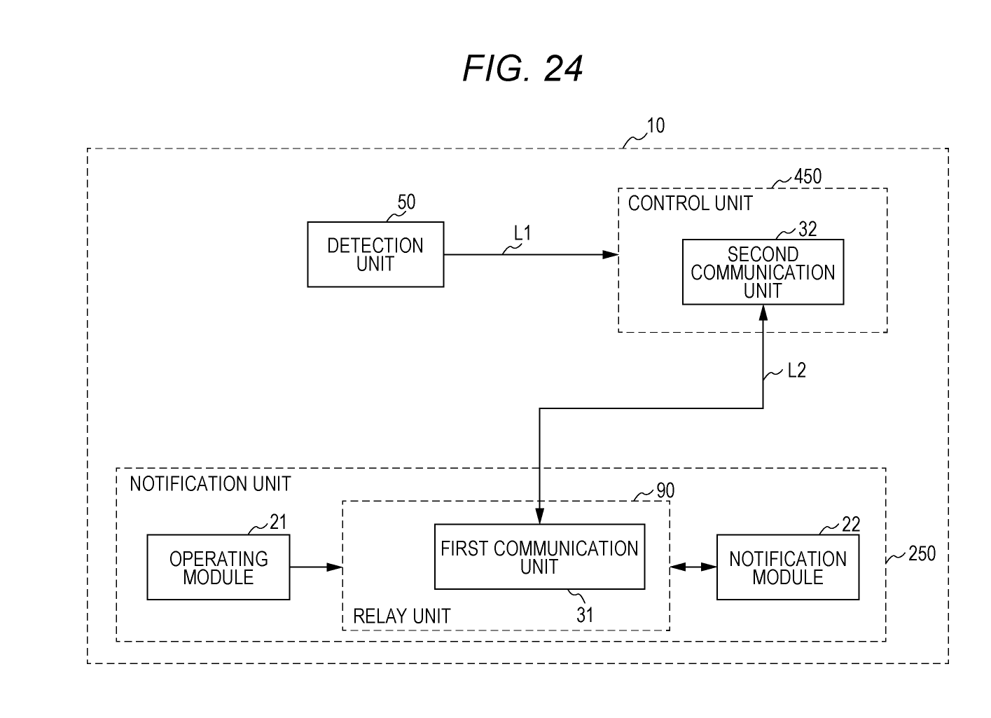

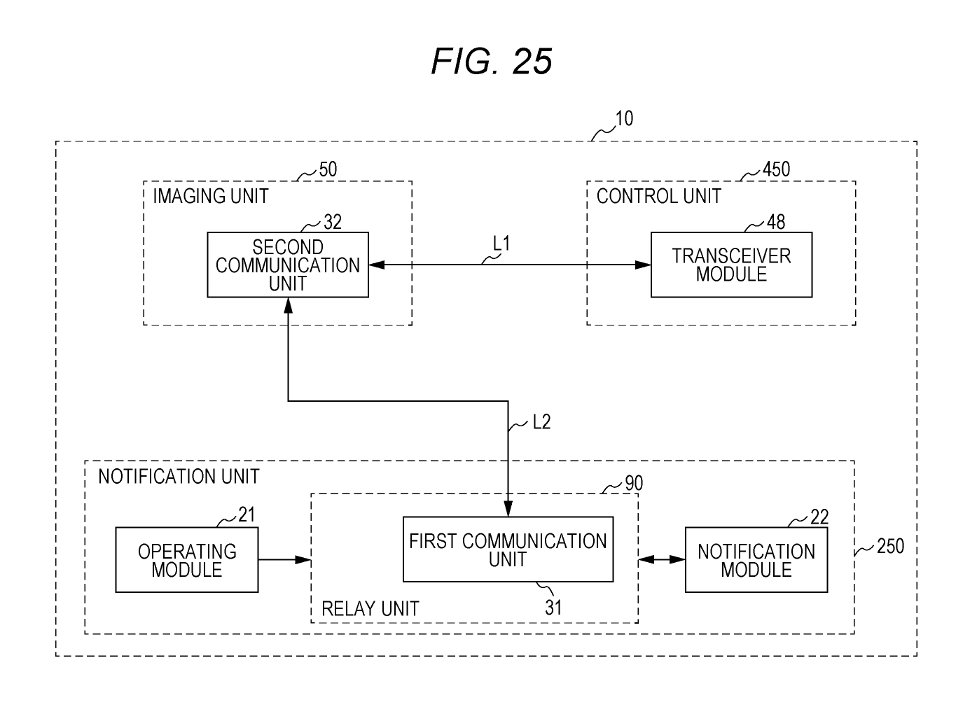

FIGS. 24 and 25 are block diagrams illustrating an example of a communication form between the control unit 450 and the relay unit 90. Although not illustrated in these drawings, the control unit 450 has the same functional configuration as that illustrated in FIG. 23, for example.

As illustrated in FIG. 24, the relay unit 90 is configured to include a first communication unit 31, and the control unit 450 is configured to include a second communication unit 32. These communication unit are connected by a communication line L2 so as to be able to transmit and receive signals, whereby the transmission and reception of signals between the control unit 450 and the relay unit 90 is enabled. In FIG. 23, the transceiver module 48 corresponds to one form of the second communication unit 32. Moreover, a transceiver module (not illustrated) provided in the relay unit 90 corresponds to one form of the first communication unit 31. The second communication unit 32 transmits and receives signals by being controlled by the control module 43, for example. The first communication unit 31 transmits and receives signals by being controlled by a control module (not illustrated) provided in the relay unit 90. The first communication unit 31 may transmit and receive signals by being controlled by the control module 43 via the second communication unit 32. In this way, the first communication unit 31 and the second communication unit 32 are configured so as to be able to communicate bidirectionally.

As illustrated in FIG. 25, the imaging unit 50 may be configured to include the second communication unit 32. The second communication unit 32 and the first communication unit 31 included in the relay unit 90 are connected by the communication line L2 so as to be able to transmit and receive signals. Moreover, the second communication unit 32 and the transceiver module 48 included in the control unit 450 are connected by the communication line L1 so as to be able to transmit and receive signals. In this way, transmission and reception of signals between the control unit 450 and the relay unit 90 is enabled via the communication line L1 and the imaging unit 50. Here, the transmission and reception via the communication line L2 may be wireless communication or cable communication.

[Overall Configuration of Nurse Call System 10]

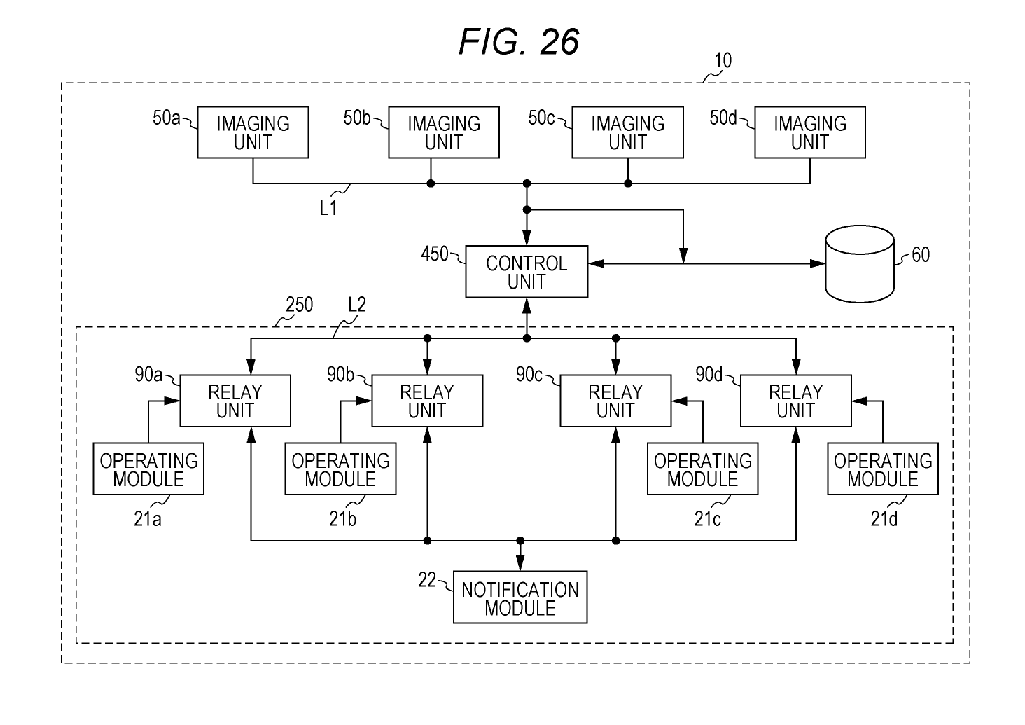

Next, an overall configuration of the nurse call system 10 of the present invention according to this embodiment will be described. FIG. 26 is a block diagram illustrating an example of an overall configuration of the nurse call system 10 according to this embodiment.

As illustrated in FIG. 26, a plurality of imaging unit 50 and the plurality of relay unit 90a to 90d are connected to the control unit 450 so as to be able to communicate with each other. The communication line L1 connects the plurality of imaging unit 50a to 50d and one control unit 450. Moreover, the communication line L2 connects the plurality of relay unit 90a to 90d and one control unit 450. Moreover, the plurality of relay unit 90a to 90d is connected to the notification module 22. The plurality of corresponding operating modules 21a to 21d is connected to each of the plurality of relay unit 90a to 90d.

The imaging unit 50a is correlated with the relay unit 90a and the operating module 21a, and these constituent elements are connected to the control unit 450 and the notification module 22 so as to be able to transmit and receive signals. In this way, the imaging unit 50a is configured similarly to the nurse call system 10 illustrated in FIG. 22. The same is true for a case where the imaging unit 50b to 50d, the relay unit 90b to 90d, and the operating modules 21b to 21d are correlated with each other.

The storage unit 60, for example, is connected to the control unit 450. The correlation information among the imaging unit 50, the relay unit 90, and the operating module 21 is stored in advance in the storage unit 60. The control unit 450 transmits the information processed based on the detection video information generated by the imaging unit 50 to the corresponding relay unit 90 based on the correlation information. Moreover, the control unit 450 can specify the imaging unit 50 and the sickbed corresponding to the notification module activation signal transmitted from the operating module 21 based on the correlation information.

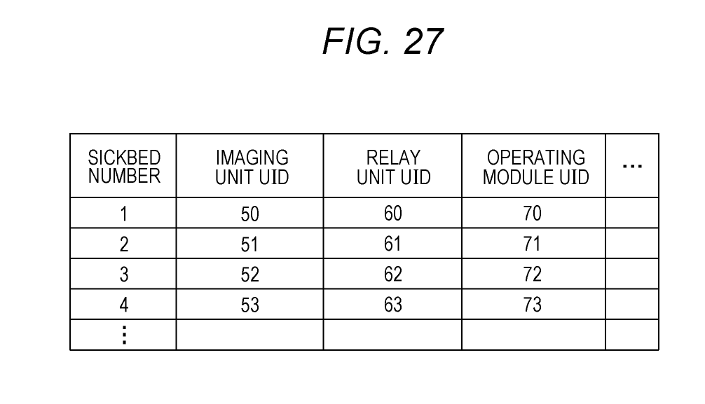

FIG. 27 is a table illustrating the imaging unit 50, the relay unit 90, and the operating module 21 corresponding to the sickbed number (sickbed UID).

As illustrated in FIG. 27, this correlation table illustrates the correlation among the sickbed number, the imaging unit UID, an UID (hereinafter sometimes referred to simply as a “relay unit UID”) applied to each relay unit 90, and the operating module UID. The information correlated with the sickbed number is not limited to these items of information, but those illustrated in the tables of FIGS. 6, 12, and 19 may be appropriately correlated with the sickbed number, for example.

When it is determined that the predetermined event has occurred based on certain detection video information, the control unit 450 can transmit the event occurrence information that specifies the sickbed to the notification module 22 based on the correlation between the detection video information and the sickbed. Specifically, the control unit 450 transmits the event occurrence information associated with the sickbed number information to the relay unit 90 specified based on such correlation as illustrated in FIG. 27. The relay unit 90 transmits the event occurrence information received from the control unit 450 to the notification module 22. The notification module 22 outputs a notification that specifies the sickbed based on the sickbed number information associated with the information. In this way, the healthcare provider having received the notification can recognize the sickbed in which the predetermined event has occurred. Moreover, the event occurrence information associated with the sickbed number information is stored in the storage unit 60. Further, for example, during determination on the occurrence of the predetermined event, the event detection video that specifies the sickbed number can be displayed on the display module 44 provided in the control unit 450. In this case, for example, the number of the corresponding sickbed number is also displayed on the display module 44 together with the event detection video.

[Hardware Configuration of Nurse Call System 10]

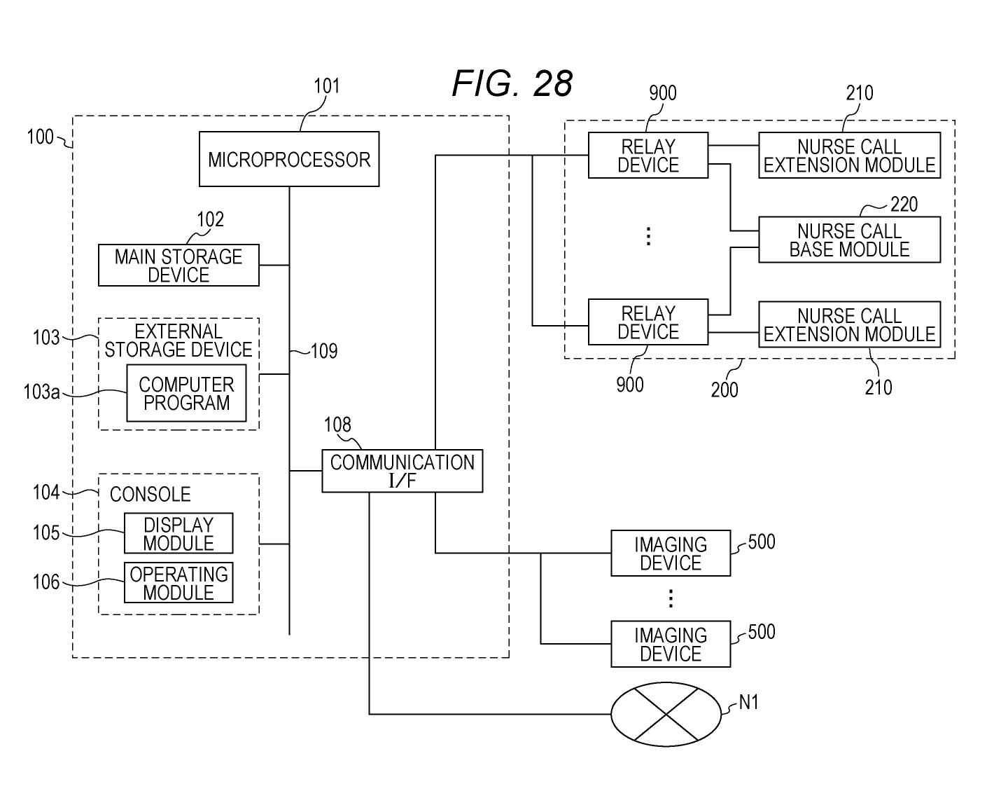

Next, a hardware configuration of the nurse call system 10 (mainly a computer 100) will be described with reference to FIG. 28. FIG. 28 is a block diagram illustrating an example of a hardware configuration of the nurse call system 10 according to this embodiment.

According to the hardware configuration of the nurse call system 10 of this embodiment, a plurality of nurse call extension modules 210 is connected to the communication interface 108 and the nurse call base module 220 via a plurality of corresponding relay devices 900.

The relay device 900 may be an arbitrary element as long as it has the above-described function of the relay unit 90. Examples of the relay device 900 include a path switcher, a signal converter, and the like.

The nurse call extension module 210 and the nurse call base module 220 are connected to the relay device 900 to form the nurse call device 200. A communication interface (not illustrated) is provided in the nurse call extension module 210 and the nurse call base module 220. In this way, the computer 100 can receive signals from the nurse call extension module 210 via the relay device 900. As a result, the computer 100 can control the nurse call base module 220 via the relay device 900.

The other constituent elements other than those described above, of the nurse call system. 10 of this embodiment may have the same configuration as those of the first to third embodiments.

[Use Form of Nurse Call System 10]

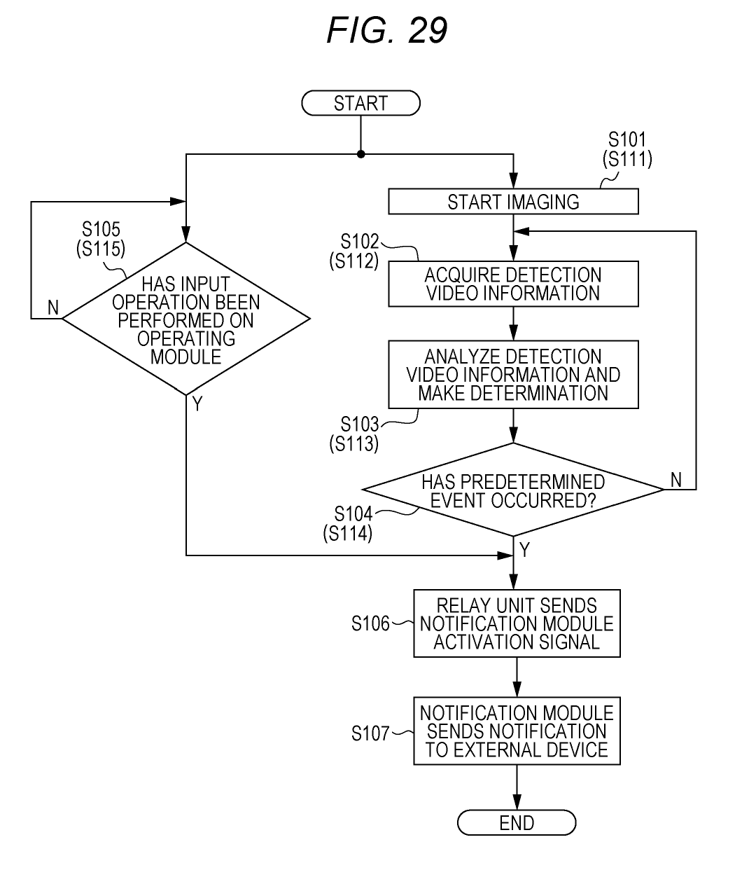

Next, a use form of the nurse call system 10 according to this embodiment will be described. FIG. 29 is a flowchart illustrating an example of a use form of the nurse call system 10 according to this embodiment. In description of the use form, the constituent elements illustrated in FIG. 24 are appropriately used.

As illustrated in FIG. 29, this flowchart illustrates processes in which the relay unit 90 transmits a notification module activation signal to the notification module 22 in a specific case by analyzing the detection video information transmitted from the imaging unit 50. Here, examples of the “specific case” include a case in which the control unit 450 determines that a predetermined event has occurred and a case in which an operation is input by the operating module 21.

First, the imaging unit 50 starts imaging a certain range including a sickbed as an imaging range, for example. With this imaging, the detection video information is continuously generated, and the detection video information is transmitted to the control unit 450 whenever it is generated (step S101).

Subsequently, the control unit 450 generates the detection video information transmitted from the imaging unit 50 (step S102).

Subsequently, the analysis module 41 of the control unit 450 analyzes the generated detection video information. The analysis result is output to the determination module 42. The determination module 42 receives the analysis result and makes determination on the occurrence of the predetermined event. This analysis is performed using the above-described algorithm or the like whereby the movement information of a subject is generated. The subject is the operator who operates the operating module 21. Subsequently, it is determined whether the generated movement information matches the movement information of a predetermined event using the above-described matching process (step S103). Moreover, as described above, an event threshold may be set according to the importance of the event, and the determination on the predetermined event is executed based on the event threshold.

When the determination module 42 determines that the predetermined event has occurred, the control module 43 controls the relay unit 90 to transmit a notification module activation signal to the notification module 22 (step S104: YES, step S106). When the determination module 42 determines that the predetermined event has not occurred, the processes of steps S102 and S103 are continued (step S104: NO, steps S102 to S103).

The relay unit 90 monitors whether an operation has been input from the operating module 21 to the notification module 22 (step S105). This process is performed in parallel with the processes of steps S101 to S104. When a notification module activation signal is input from the operating module 21 to the relay unit 90, the relay unit 90 transmits the notification module activation signal to the notification module 22 (step S105: YES, step S106). The notification module activation signal is input to the notification module 22 when the operating module 21 inputs an operation. On the other hand, when the notification module activation signal is not input to the relay unit 90, the monitoring is performed continuously (step S105: NO).

Upon receiving the notification module activation signal from the relay unit 90, the notification module 22 outputs a notification to the external device using a notification buzzer or the like provided in the notification module 22 (step S107), and the process ends.

When the process of step S106 is performed, the transmission history of the notification module activation signal from the relay unit 90 to the notification module 22 is recorded on the storage unit 60 or the like. In this case, if the process of step S106 results from the process of step S105, information such as the UID of the operating module used for inputting the operation and the date and time when the operation was input is stored in association with the transmission history. If the process of step S106 results from the process of step S104, information such as the UID of the imaging unit 50, the type of the predetermined event, and the date and time when the event occurred is stored in association with the transmission history. If the process of step S106 results from the processes of steps S104 and S105, information such as the UID of the operating module 21, the UID of the imaging unit 50, the type of the predetermined event, the date and time when the operation was input, and the date and time when the event occurred is stored in association with the transmission history. Moreover, in this case, the occurrence history of the predetermined event may be selectively stored.

This flowchart illustrates the processes performed until the notification module 22 outputs a notification to the external device when the control unit 450 receives the notification module activation signal. However, this embodiment is not limited to this, but the imaging unit 50 may continuously generate the detection video information even when the notification module activation signal is received, for example. Moreover, whenever the notification module activation signal is received, the flow may proceed to the process routine of steps S104 to S107.

[Effects of Nurse Call System 10]

According to the nurse call system 10 of this embodiment, the control unit 40 of the nurse call system according to any one of the first to third embodiments is replaced with the relay unit 90, and the control unit 450 connected to the relay unit 90 is added. The imaging unit 50 is connected to the control unit 450. The control unit 450 can have the same configuration as the control unit 40. Thus, the same effects as those of the first to third embodiments can be obtained.

In the notification unit 250 of the nurse call system 10 of this embodiment, the relay unit 90 is provided between the operating module 21 and the notification module 22. Further, the operating module 21 and the notification module 22 can communicate with each other via the relay unit 90. Thus, a conventional notification device can be easily modified to the nurse call system 10 of the present invention. Specifically, the notification unit 250 of the present invention can be formed by providing the relay unit 90 on a communication line path of the conventional notification device in which the operating module 21 and the notification module 22 are connected by a communication line. In this way, the nurse call system 10 of the present invention can be constructed by utilizing the nurse call base module and the nurse call extension module of the nurse call device which is already installed in a hospital, for example.

As a result, an existing nurse call device can be utilized in a hospital or the like in which the nurse call device is already installed. That is, it is possible to simplify a construction work as compared to when a new nurse call system is constructed by replacing the existing nurse call device. Moreover, the cost associated with the construction can be reduced.

Since the control unit 450 is configured to be independent from the notification unit 250, the specification of the nurse call system 10 of the present invention can be changed easily by replacing the control unit 450 even after the construction.

### Fifth Embodiment

### Nurse Call System 10

A configuration of a nurse call system according to this embodiment will be described with reference to the drawings. The description will be given in the following order: Functional configuration of nurse call system; Overall configuration of nurse call system; and Hardware configuration for realizing functional configuration.

[Functional Configuration of Nurse Call System. 10]

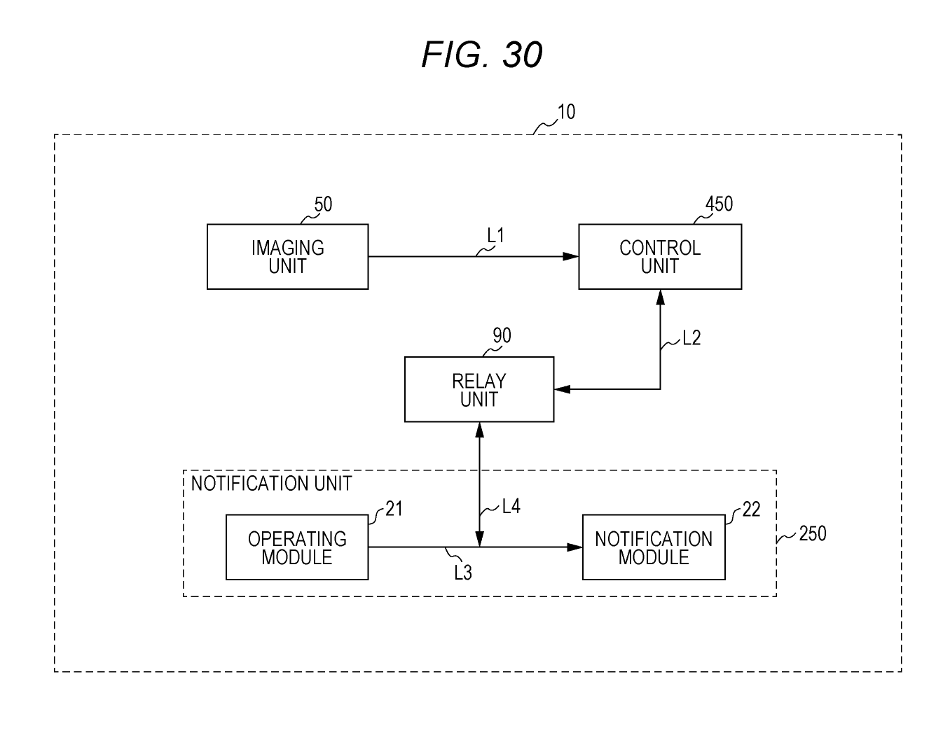

FIG. 30 is a functional block diagram illustrating an example of a nurse call system according to this embodiment. As illustrated in FIG. 30, the nurse call system 10 has a configuration in which the notification unit 250 is connected by a communication line L3 so that information (signals) can be transmitted at least from the operating module 21 to the notification module 22.

(Relay Unit 90)

The relay unit 90 is provided outside the notification unit 250. The relay unit 90 is connected to the communication line L3 via a communication line L4. The communication lines L3 and L4 are configured so that information can be transmitted at least from the operating module 21 to the relay unit 90 and information can be transmitted and received between the relay unit 90 and the notification module 22. According to an example of such a configuration, the communication line L4 may branch halfway from the communication line L3 so as to extend toward the relay unit 90. The other constituent elements are the same as those of the nurse call system of the fourth embodiment. The communication via the communication lines L3 and L4 may be wireless communication or cable communication, which can be selected appropriately as necessary.

The signals transmitted from the relay unit 90 to the notification unit 250 via the communication line L4 are input to the notification module 22 via the communication line L3. Moreover, the notification module activation signal transmitted from the operating module 21 is input to the notification module 22 via the communication line L3 and is also input to the relay unit 90 via the communication line L4. That is, the communication line L4 has a function of squeezing signals into the communication line L3.

The communication line L4 may be connected directly to the operating module 21, for example. In this way, the control unit 450 can directly control the operating module 21 via the relay unit 90. In this case, the operating module 21 can transmit the notification module activation signal to the notification module 22. Moreover, the communication line L4 may be connected directly to the notification module 22, for example. In this case, the control unit 450 can directly control the notification module 22 via the relay unit 90.

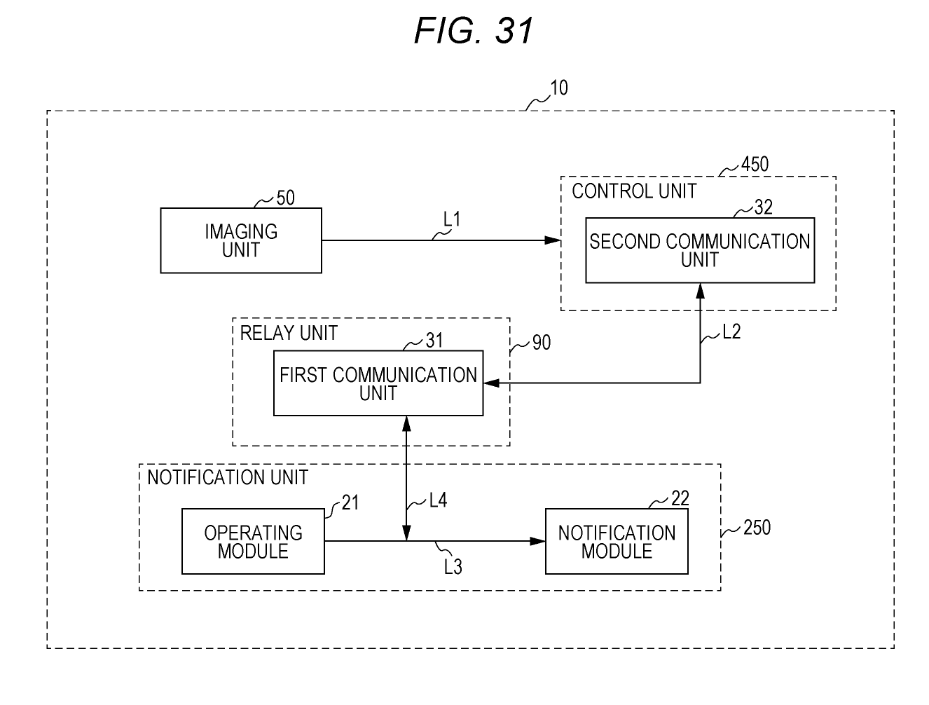

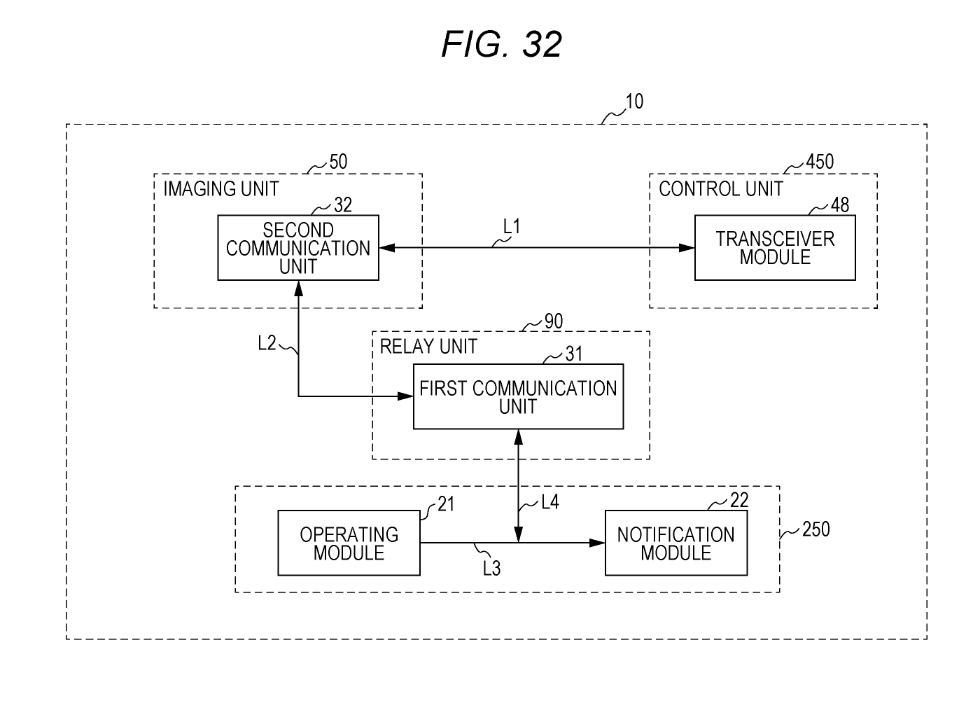

FIG. 31 is a functional block diagram illustrating an example of a communication form between the control unit 450 and the relay unit 90. FIG. 32 is a functional block diagram illustrating an example of a communication form between the relay unit 90 and the notification unit 250. As illustrated in FIGS. 31 and 32, the first communication unit 31 is connected via the communication lines L3 and L4 so as to be able to communicate with the notification unit 250. In this way, when the notification module activation signal is transmitted from the control unit 450, the first communication unit 31 can transmit the notification module activation signal to the notification module 22 via the communication lines L3 and L4.

[Overall Configuration of Nurse Call System 10]

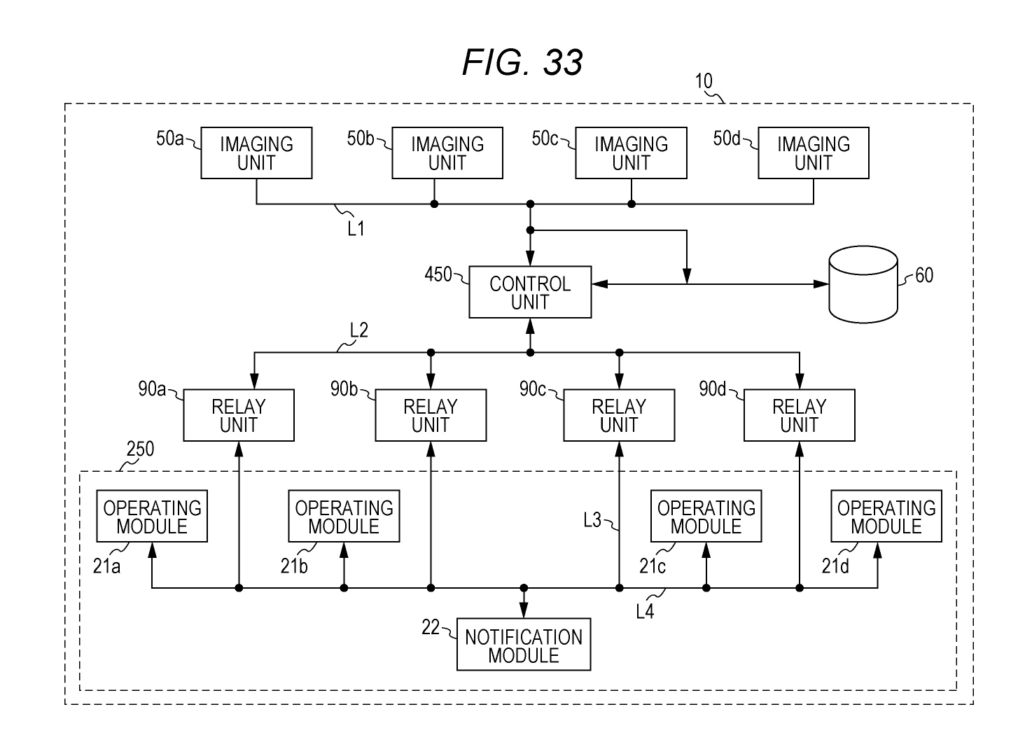

Next, an overall configuration of the nurse call system 10 according to this embodiment will be described. FIG. 33 is a block diagram illustrating an example of an overall configuration of the nurse call system 10 according to this embodiment.

As illustrated in FIG. 33, a plurality of operating modules 21a to 21d and the notification module 22 are connected by the communication line L4. A plurality of relay unit 90a to 90d is connected to the communication line L4 via the communication line L3.

The imaging unit 50a is correlated with the relay unit 90a and the operating module 21a. These constituent elements are connected to the control unit 450 and the notification module 22 so as to be able to output and input information similarly to the configuration of the above-described embodiments. In this way, the nurse call system 10 of this embodiment is configured similarly to the nurse call system 10 illustrated in FIG. 30. The same is true for a case where the imaging unit 50b to 50d are correlated with the relay unit 90b to 90d and the operating modules 21b to 21d.

The information processed by the control unit 450 can be transmitted to the corresponding relay unit 90 based on the correlation information among the imaging unit 50, the relay unit 90, and the operating module 21. This information is the information which is processed based on the detection video information generated by the imaging unit 50. Moreover, when the notification module activation signal is transmitted from the operating module 21, the identification information of the operating module 21 is associated with this signal. The control unit 450 can specify the relay unit 90, the imaging unit 50, and the sickbed corresponding to the signal based on the correlation information. The information correlated with the sickbed number is not limited to these items of information, but those illustrated in the tables of FIGS. 6, 12, 19, and 27 may be appropriately correlated with the sickbed number, for example.

[Hardware Configuration of Nurse Call System 10]

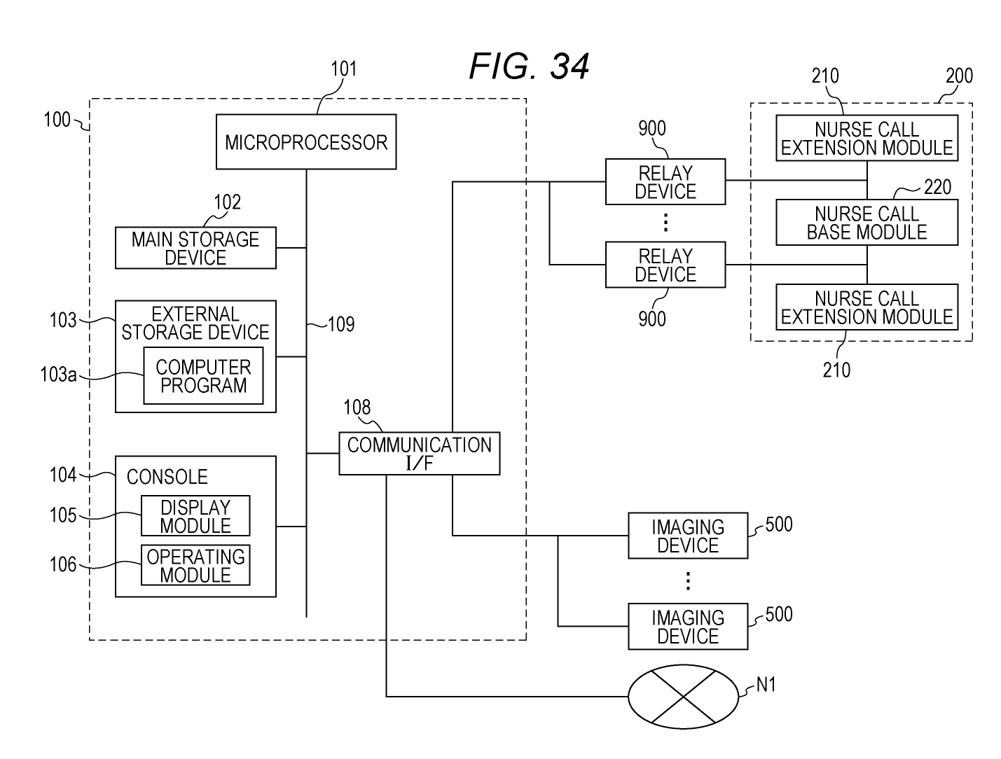

Next, a hardware configuration of the nurse call system 10 (mainly a computer 100) of the present invention, for realizing the above-described functions will be described with reference to FIG. 34. FIG. 34 is a block diagram illustrating an example of a hardware configuration of the nurse call system 10 according to this embodiment.

The hardware configuration of the nurse call system 10 according to this embodiment is the same as that of the nurse call system 10 of the present invention according to the third embodiment except that the nurse call extension module 210 and the nurse call base module 220 are connected by a communication line to form the nurse call device 200 and the relay device 900 is connected to the communication line.

[Use Form of Nurse Call System 10]

The use form of the nurse call system 10 of this embodiment is the same as the use form of the nurse call system 10 of the third embodiment.

[Effects of Nurse Call System 10]

In the nurse call system 10 of this embodiment, the notification unit 250 and the control unit 450 which are formed by connecting the operating module 21 and the notification module 22 can transmit and receive signals via the relay unit 90 connected to the notification unit 250. Thus, the same effects as the fourth embodiment can be obtained. Moreover, the nurse call system 10 of this embodiment provides the relay unit 90 using the communication line L4 having the function of squeezing signals into the communication line L3. Thus, the conventional notification device of the nurse call system can be used as it is as the notification unit 250 that forms the nurse call system 10 of the present invention. As a result, when the present embodiment is applied to an existing nurse call device installed in advance, it is possible to further simplify the construction work and to further reduce the cost associated with the construction.

### Sixth Embodiment

### Nurse Call System 10

A configuration of a nurse call system according to this embodiment will be described with reference to the drawings. The description will be given in the following order: Functional configuration of nurse call system; Overall configuration of nurse call system; and Hardware configuration for realizing functional configuration.

[Functional Configuration of Nurse Call System 10]

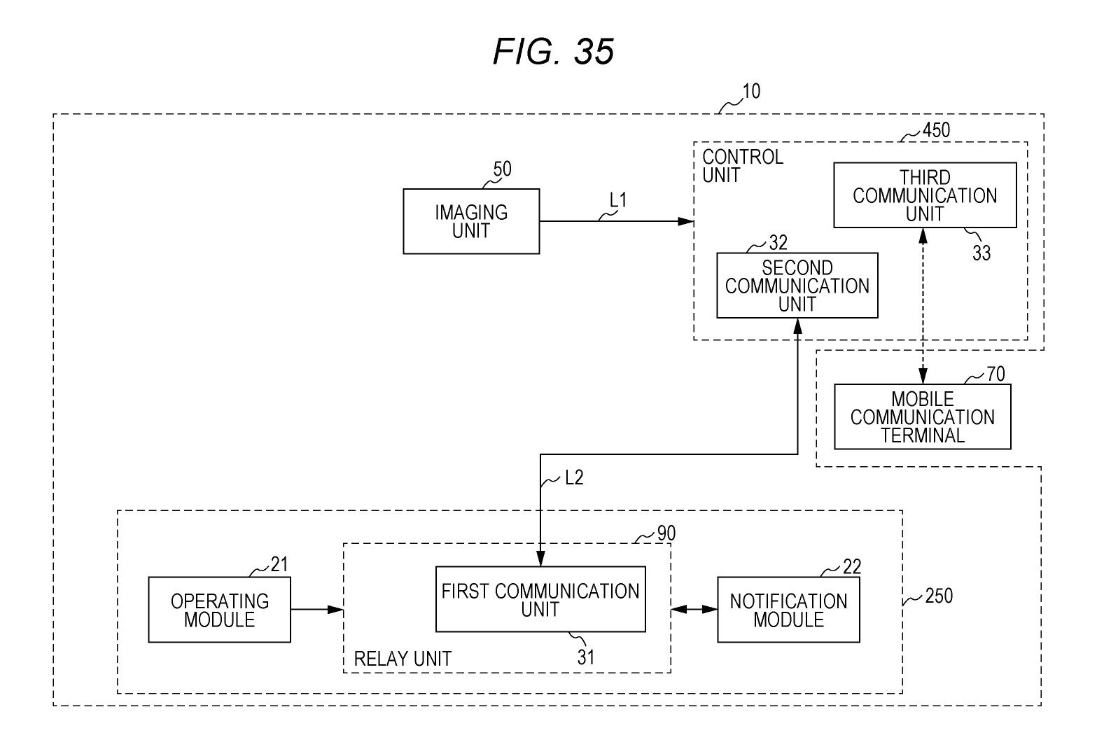

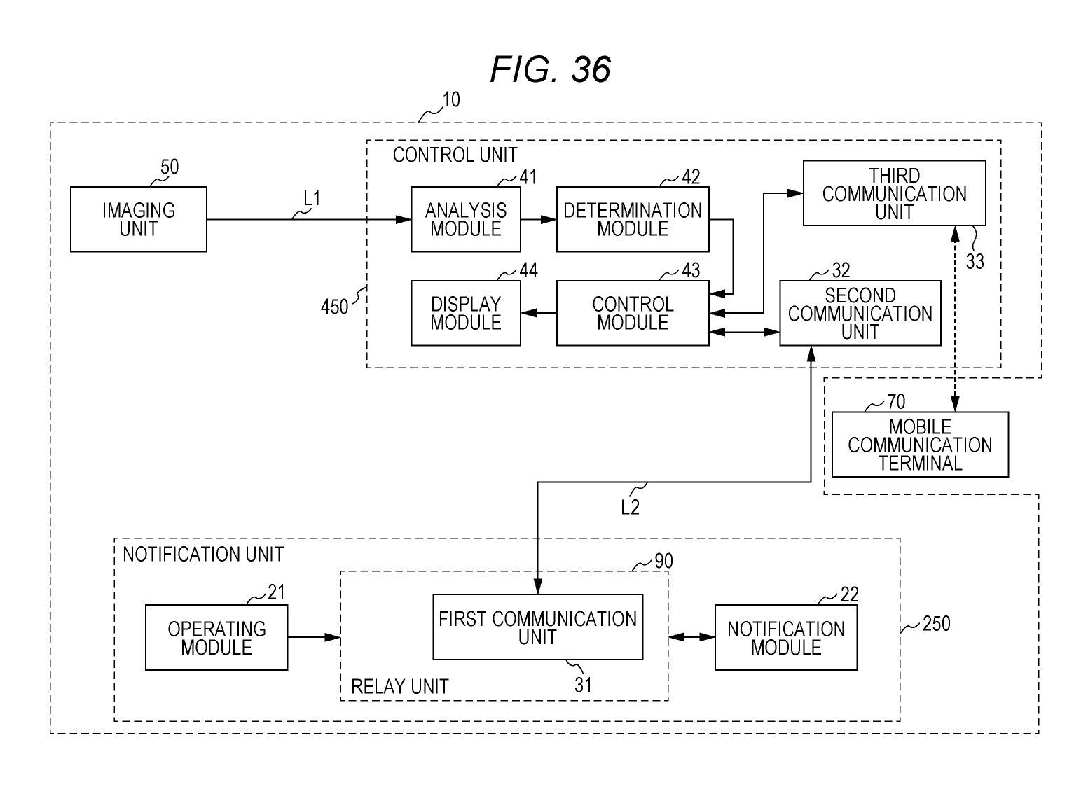

FIGS. 35 and 36 are functional block diagrams illustrating an example of a nurse call system according to this embodiment. FIG. 36 illustrates an example of a detailed configuration of the nurse call system 10 illustrated in FIG. 35.

As illustrated in FIG. 36, the control unit 450 of the nurse call system 10 of the present invention includes a third communication unit capable of transmitting and receiving signals to and from the mobile communication terminal 70. The mobile communication terminal 70 can be configured as described in the second embodiment, for example. Moreover, the third communication unit 33 may be configured as described in the second embodiment by substituting the control unit 40 with the control unit 450.

The third communication unit 33 transmits information indicating that “the notification module activation signal has been output to the notification module 22” to the mobile communication terminal 70. This signal is input to the control unit 450 via the relay unit 90 when the notification module activation signal is transmitted according to the operation of the operating module 21, for example. The control unit 450 having received the signal transmits information indicating that a “call operation has been performed on the operating module 21” from the third communication unit 33 to the mobile communication terminal 70 (notification of call operation). Moreover, when it is determined that the predetermined event has occurred, the control unit 450 controls the relay unit 90. In this case, the control unit 450 transmits the event occurrence information to the mobile communication terminal 70 via the third communication unit 33 (notification of occurrence of event). Moreover, when the second communication unit 32 has a wireless communication function, the second communication unit 32 may perform the function of the third communication unit 33.

When the mobile communication terminal 70 has the display module 70B, the event detection video is displayed on the display module 70B. The items of information described above, for example, can be appropriately selected and used for the information displayed on the display module 70B. In this case, the display module 70B corresponds to a “fourth display module” of the present invention.

[Overall Configuration of Nurse Call System 10]

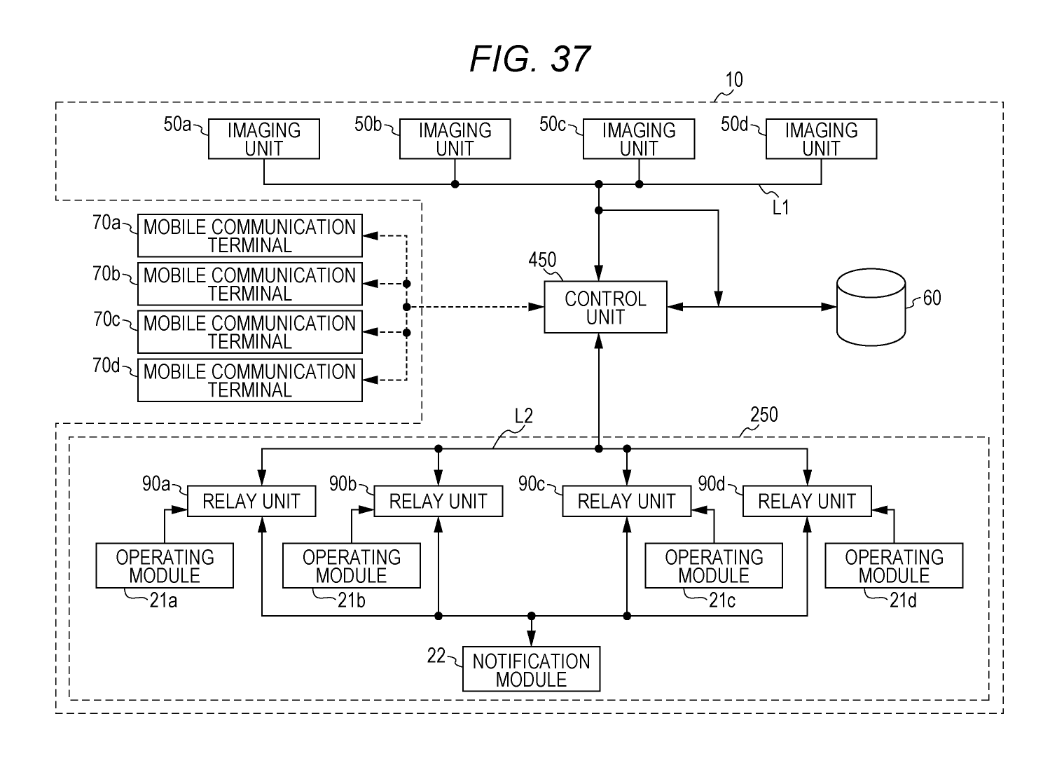

Next, an overall configuration of the nurse call system 10 according to this embodiment will be described. FIG. 37 is a block diagram illustrating an example of an overall configuration of the nurse call system 10 according to this embodiment.

As illustrated in FIG. 37, a plurality of mobile communication terminals 70a to 70d are connected to the control unit 450. The mobile communication terminal 70a is correlated with the imaging unit 50a, the relay unit 90a, and the operating module 21a and these constituent elements are connected to the control unit 450 so as to be able to communicate with each other. In this way, the mobile communication terminal 70a is configured similarly to the nurse call system 10 of the present invention illustrated in FIG. 35. The same is true for the mobile communication terminals 70b to 70d.

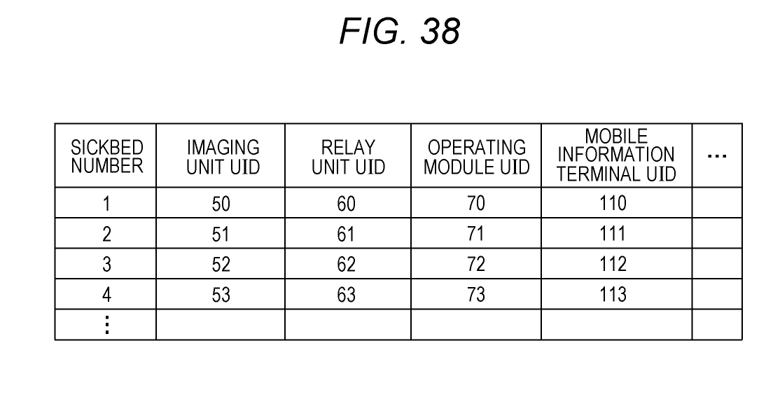

FIG. 38 is a table illustrating the imaging unit 50, the relay unit 90, the operating module 21, and the mobile communication terminal 70 corresponding to the sickbed number (sickbed UID). For example, in this table, similarly to the examples illustrated in FIG. 6 and other drawings, UIDs may be applied to the patient and the healthcare provider and may be correlated with the sickbed number to form the correlation information.

As illustrated in FIG. 38, this correlation table illustrates the sickbed number, the imaging unit UID, the relay unit UID, the operating module UID, and the mobile communication terminal UID. The information correlated with the sickbed number is not limited to these items of information, but those illustrated in the tables of FIG. 6 and other drawings may be appropriately correlated with the sickbed number, for example. For example, when a healthcare provider (a physician in charge, a nurse in charge, or the like) who preferentially rushes to a specific patient is determined, a UID may be applied to such a person in charge, a mobile information terminal carried by the person in charge may be selected, and information may be output to the mobile information terminal.

Since the correlation between the imaging unit 50 and the mobile communication terminal 70 can be identified from this table, for example, when a predetermined event is determined based on predetermined detection video information, the corresponding mobile communication terminal UID is specified from the information (the imaging unit UID) of the imaging unit 50 associated with the detection video information. The control unit 450 can notify the specified mobile communication terminal 70 of the determination result and display a live video, an event detection video, or the like on the display module 70B provided in the mobile communication terminal 70 as necessary. Moreover, since the correlation between the operating module 21 and the mobile communication terminal 70 can be identified from the table, for example, when the operating module 21 is operated, the control unit 450 can transmit information indicating that the operating module 21 was “operated” to the corresponding mobile communication terminal 70 with the notification module activation signal and can display the information on the display module 70B provided in the mobile communication terminal 70 as the event detection video as necessary.

[Hardware Configuration of Nurse Call System 10]

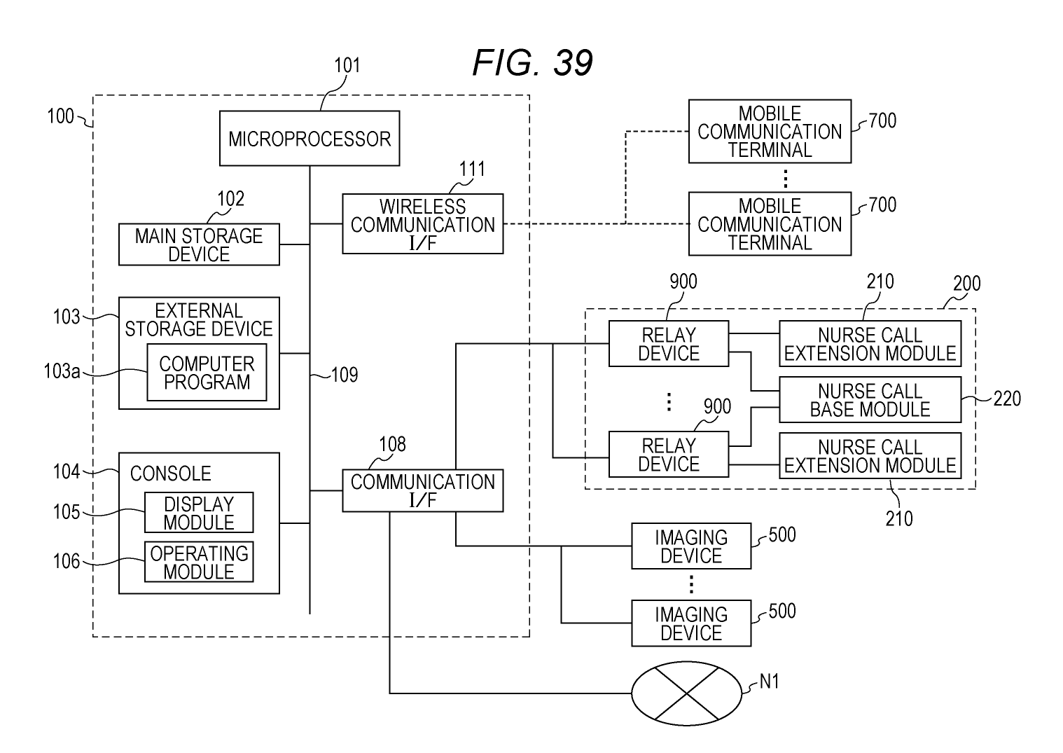

Next, a hardware configuration of the nurse call system 10 (mainly a computer 100) of the present invention, for realizing the above-described functions will be described with reference to FIG. 39. FIG. 39 is a block diagram illustrating an example of a hardware configuration of the nurse call system 10 according to this embodiment.

According to the hardware configuration of the nurse call system 10 of this embodiment, a plurality of mobile communication terminals 700 is connected via the wireless communication interface (I/F) 111 so as to be able to communicate with each other. The wireless communication interface 111 can be configured as described in the second embodiment, for example.

The other constituent elements other than those described above, of the nurse call system 10 of this embodiment may have the same configuration as those of the first to fifth embodiments.

[Use Form of Nurse Call System 10]

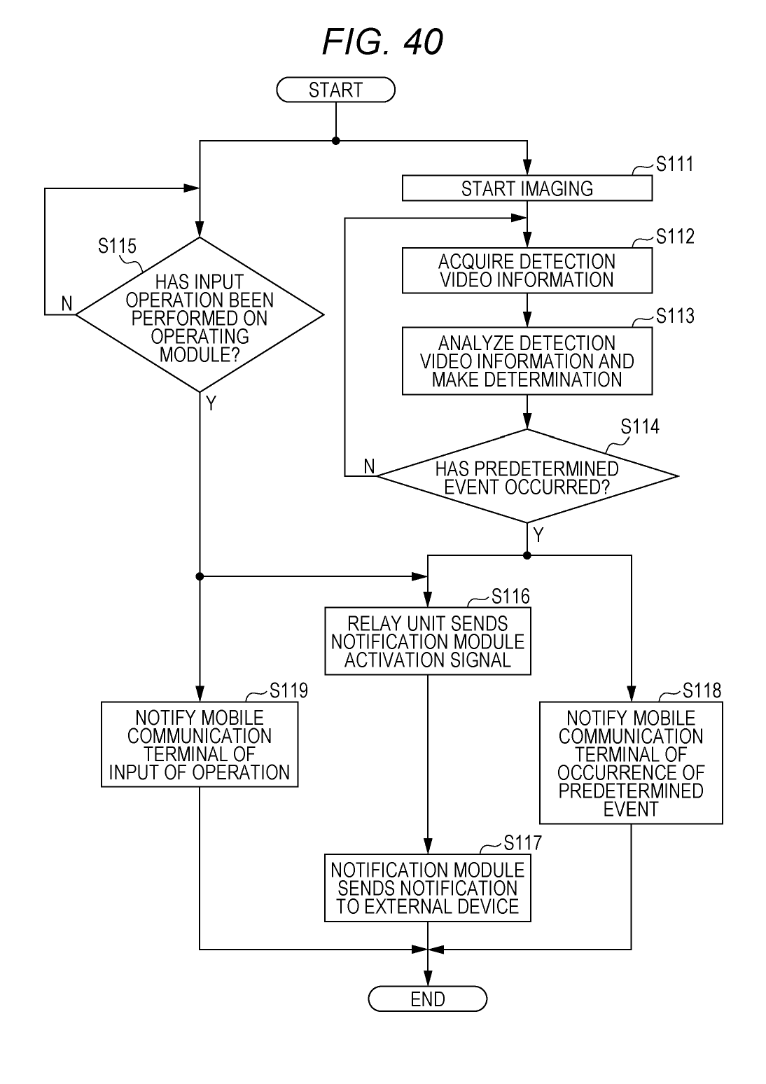

Next, a use form of the nurse call system 10 according to this embodiment will be described. FIG. 40 is a flowchart illustrating an example of a use form of the nurse call system 10 according to this embodiment. In description of the use form, the constituent elements illustrated in FIG. 36 are appropriately used.

As illustrated in FIG. 40, this flowchart illustrate processes in which, when the control unit 450 determines that a predetermined event has occurred or the control unit 450 receives the notification module activation signal, information indicating the notice thereof is transmitted to the mobile communication terminal 70 in addition to the processes of the flowchart illustrated in FIG. 29. Hereinafter, the respective steps will be described in detail.

The processes of steps S111 to S115 are performed similarly to the processes of steps S101 to S105 of the flowchart illustrated in FIG. 29 (steps S111 to S115).

When the determination module 42 determines that the predetermined event has occurred (step S114: YES), the control module 43 controls the relay unit 90 to transmit the notification module activation signal to the notification module 22 (step S116). The control module 43 outputs a notification of the event occurrence information to the mobile communication terminal 70 (step S118). The mobile communication terminal 70 informs the external device of the occurrence of the predetermined event in response to the notification. This notification may be realized in an arbitrary form as long as it can be sensed by the five senses of a person and may be realized via sound, characters, videos, vibration, and the like, for example. When it is determined that the predetermined event has not occurred (step S114: NO), the processes of steps S112 and S113 are continued (steps S112 to S113).

When the notification module activation signal from the operating module 21 is input to the relay unit 90 (step S115: YES), the relay unit 90 transmits the notification module activation signal to the notification module 22 (step S116). The relay unit 90 further notifies the mobile communication terminal 70 of the fact that an input operation was performed on the operating module 21 (step S119). The mobile communication terminal 70 notifies the external device of the information on the input operation in response to the notification. When the notification module activation signal from the operating module 21 is not input to the relay unit 90, the monitoring is continued (step S115: NO).

Upon receiving the notification module activation signal from the relay unit 90, the notification module 22 outputs a notification to the external device using a notification buzzer or the like provided in the notification module 22, and the process ends (step S117).

Although this flowchart illustrates the processes performed until the notification module 22 outputs a notification to the external device when the control unit 450 receives the notification module activation signal. The imaging unit 50 may continuously generate the detection video information even when the notification module activation signal is received. Moreover, whenever the notification module activation signal is received, the flow may proceed to the process routine of steps S114 to S119.

The other features of the use form of the nurse call system 10 of the present invention according to this embodiment are the same as those of the use form of the nurse call system 10 according to the fourth and fifth embodiments.

[Effects of Nurse Call System 10]

According to the nurse call system 10 of this embodiment, the notification unit 250 and the control unit 450 which are formed by connecting the operating module 21 and the notification module 22 can transmit and receive signals via the relay unit 90. Thus, the same effects as the fourth and fifth embodiments can be obtained.

According to the nurse call system 10 of this embodiment, since the third communication unit that can communicate with the mobile communication terminal 70 is provided in the control unit 450, when the control unit 450 determines that a predetermined event has occurred or the control unit 450 receives the notification module activation signal, information indicating the notice thereof can be transmitted to the mobile communication terminal 70. Due to this, for example, even when a healthcare provider in a hospital is not present in a nurse's station, the healthcare provider can be informed of an abrupt change in the condition of the patient through the mobile communication terminal 70. Moreover, when the mobile communication terminal 70 has a display module, the video during the occurrence of a predetermined event is displayed on the display module, and a quick response such as treatment can be performed.

While embodiments of the present invention have been described in detail, the present invention is not limited to the above-described embodiments, and various modifications based on the technical ideal of the present invention can be made. Moreover, some or all of the embodiments may be appropriately combined to form another embodiment. The nurse call system is one form of the notification system, and the same configuration can be applied to a caring system, a home nursing system, and the like.

The numbers, structures, configurations, shapes, materials, and the like mentioned in the above-described embodiments are examples only, and other numbers, structures, configurations, shapes, materials, and the like different from those above may be used.

### REFERENCE SIGNS LIST

10: Nurse call system
20, 250: Notification unit
21: Operating module
22: Notification module
30: Wireless communication unit
40, 450: Control unit
41: Analysis module
42: Determination module
43: Control module
46: Storage module
47: Storage priority application module
50: Imaging unit
60: Storage unit
70: Mobile communication terminal
80: Microwave Doppler sensor
90: Relay unit

1. A notification system comprising:
a detection unit that generates movement information of a subject according to a change over time;
a storage unit that is connected to the detection unit and stores the movement information; and
a control unit that receives the movement information from the detection unit, analyzes the movement information, makes a determination on the occurrence of a predetermined event from the result of the analysis, specifies the movement information in a predetermined period including the time when the determination was made, applies a storage priority value to the movement information according to a type of the predetermined event occurred, and transmits the movement information to the storage unit.

2. The notification system according to claim 1, wherein
the control unit performs the specification using a period that starts a first predetermined period earlier than the time when the determination was made and ends a second predetermined period later than the time when the determination was made as the predetermined period.

3. The notification system according to claim 1 or 2, further comprising:
a notification module configured to output a notification to an external device, wherein
the control unit transmits, to the notification module, the movement information of the subject in the predetermined period including the time when the determination was made when it is determined that the predetermined event has occurred.

4. The notification system according to any one of claims 1 to 3, wherein
the detection unit is an imaging unit configured to be capable of perform imaging,
the imaging unit generates video information including the movement information of the subject, and
the control unit includes a first display module configured to be able to display a video and analyzes the video information, and when the presence of a predetermined movement of the subject is determined as the occurrence of the predetermined event based on a change in a luminance value of a pixel that forms the video, the control unit displays a video imaged by the imaging unit in the predetermined period on the first display module according to the result of the determination.

5. The notification system according to claim 4, wherein
a video indicating a predetermined movement of the subject is a video imaged by the imaging unit in a predetermined period including the time of the determination, stored in the storage unit.

6. The notification system according to claim 3, wherein
the detection unit is a microwave Doppler sensor that includes a transmitting module that emits microwaves to an area including the chest of the subject and a receiving module that receives reflection waves from the subject as the movement information,
the control unit analyzes a respiratory state of the subject based on a Doppler shift of the reflection waves,
the control unit determines whether the respiratory state has reached a predetermined respiratory state as the occurrence of the predetermined event based on the result of the analysis, and
the control unit transmits control information of the notification module when the predetermined respiratory state is reached.

7. The notification system according to any one of claims 1 to 6, wherein
the control unit removes the movement information of the subject stored in the storage unit under a predetermined condition, and the removal is executed in ascending order of the storage priority values.

8. The notification system according to claim 7, wherein
the control unit performs the removal when a predetermined period has elapsed from the storage in the storage unit or when an available volume of the storage unit is lower than a predetermined value as the predetermined condition.

9. The notification system according to any one of claims 1 to 8, further comprising:
a transceiving unit configured to be able to transmit information to at least one mobile communication terminal, wherein
the control unit transmits information based on the occurrence of the predetermined event specified by the determination to the mobile communication terminal via the transceiving unit.

10. The notification system according to claim 9, wherein
the mobile communication terminal includes a second display module configured to be able to display information based on the movement information, and
the control unit transmits the storage priority value and the information based on the movement information corresponding to the predetermined event specified by the determination to the mobile communication terminal via the transceiving unit, and the mobile communication terminal displays the information on the second display module.

11. The notification system according to claim 4, further comprising:
a transceiving unit configured to be able to transmit a video to at least one mobile communication terminal, wherein
the mobile communication terminal includes a second display module configured to be able to display the video,
the control unit transmits a live video of the subject being imaged by the imaging unit to the mobile communication terminal via the transceiving unit when a predetermined movement of the subject has occurred, and
the mobile communication terminal is configured so that a terminal operator who operates the mobile communication terminal can make communication or a telephone call with the subject while viewing the live video by performing a predetermined operation.

12. The notification system according to any one of claims 9 to 11, wherein
the subject and the mobile communication terminal are correlated and the transceiving unit is configured to be able to transmit information to a specific mobile communication terminal,
the detection unit is correlated with the subject,
upon receiving the movement information of the subject from the detection unit, the control unit specifies the subject based on the correlation between the detection unit and the subject, specifies the mobile communication terminal to which information is to be transmitted based on the correlation between the subject and the mobile communication terminal, and transmits the movement information of the subject to the specified mobile communication terminal via the transceiving unit.

13. A notification system comprising:
a notification unit including an operating module configured to be able to input operations and a notification module disposed at a position separated from the operating module and configured to be able to output a notification to an external device, the notification unit being configured such that the notification module operates to output a notification to the external device in response to a first signal for operating the notification module, transmitted from the operating module when an operator inputs an operation;
a detection unit that generates movement information of the operator according to a change over time;
a control unit that receives the movement information from the detection unit, analyzes the movement information, and makes a determination on the occurrence of a predetermined event from the result of the analysis; and
a relay unit that is provided between the operating module and the notification module and is configured to allow the notification module and the control unit to communicate with each other, the relay unit transmitting the first signal to the notification module, wherein
the control unit transmits a second signal to the relay unit when the determination is executed, and the relay unit transmits a third signal for operating the notification module to the notification module in response to the second signal.

14. The notification system according to claim 13, further comprising:
a first communication unit provided in the relay unit; and
a second communication unit provided in the detection unit or the control unit, wherein
the first communication unit and the second communication unit communicate bidirectionally whereby the notification module can communicate with the detection unit or the control unit bidirectionally via the relay unit.

15. The notification system according to claim 14, wherein
when it is determined that the predetermined event has occurred, the control unit transmits the second signal to the relay unit via the second communication unit and the first communication unit.

16. The notification system according to claim 14 or 15, wherein
the first communication unit and the second communication unit are wireless communication unit.

17. The notification system according to any one of claims 13 to 16, wherein
the detection unit is an imaging unit configured to be able to generate videos, and
the control unit includes a third display module configured to be able to display videos and displays a video imaged by the imaging unit in a predetermined period including the time when the determination was made or the input operation was executed on the third display module when it is determined that the predetermined event has occurred or the input operation was performed.

18. The notification system according to claim 17, wherein
the control unit determines the occurrence of the predetermined event based on a change in a luminance value of a pixel that forms the video in response to a predetermined movement of the operator.

19. The notification system according to claim 16, wherein
the control unit includes a third communication unit configured to be able to communicate with at least one mobile communication terminal,
when it is determined that the predetermined event has occurred, the control unit transmits information based on the result of the determination to the mobile communication terminal via the third communication unit, and
the relay unit notifies the mobile communication terminal of information indicating that the third signal has been transmitted via the first communication unit.

20. The notification system according to claim 19, wherein
the mobile communication terminal includes a fourth display module configured to be able to display videos, and
the control unit transmits a video corresponding to the predetermined event occurred to the mobile communication terminal via the second communication unit or the third communication unit together with the information indicating the execution of the determination, and the mobile communication terminal displays the video on the fourth display module.

21. The notification system according to any one of claims 13 to 20, wherein
the notification unit is a nurse call system which includes a switch which is the operating module and a call module which is the notification module and in which the call module operates when the operator operates the switch.
